(19) United States
(12) Patent Application Publication
(10) Pub. No.: US 2002/0147331 A1
(43) Pub. Date: Oct. 10, 2002

(54) Methods for synthesis of oligonucleotides

(76) Inventors: Andrei P. Guzaev, Carlsbad, CA (US); Muthiah Manoharan, Carlsbad, CA (US)

Correspondence Address: Michael P. Straher, Esq., Woodcock Washburn Kurtz, Mackiewicz & Norris LLP, One Liberty Place - 46th Floor, Philadelphia, PA, 19103, US

(21) Appl. No.: 09/775,967

(22) Filed: Feb. 2, 2001

(51) Int. Cl. C07H 21/04; C08F 2/00

(52) U.S. Cl. 536/25.3; 525/54.2

(57) ABSTRACT

The present invention is directed to improved methods and compositions for synthesis of oligonucleotides and other phosphorus-linked oligomers, without the need for phosphoryl protecting groups. The methods involve the reaction of nucleoside phosphoramidites with a support-bound oligomer having one or more unprotected phosphorus-containing internucleoside linkages in the presence of a neutralizing agent.

### FIELD OF THE INVENTION

The present invention is directed to improved methods and compositions for synthesis of oligonucleotides and other phosphorus-linked oligomers, without the need for phosphoryl protecting groups. Oligomers synthesized using the methods of the invention are useful for diagnostic reagents, research reagents and in therapeutics.

### BACKGROUND OF THE INVENTION

It is well known proteins are significantly involved in many of the bodily states in multicellular organisms, including most disease states. Such proteins, either acting directly or through their enzymatic or other functions, contribute in major proportion to many diseases and regulatory functions in animals and man. For disease states, classical therapeutics has generally focused upon interactions with such proteins in efforts to moderate their disease-causing or disease-potentiating functions. In newer therapeutic approaches, modulation of the production of such proteins is desired. By interfering with the production of proteins, the maximum therapeutic effect might be obtained with minimal side effects. It is the general object of such therapeutic approaches to interfere with or otherwise modulate gene expression which would lead to undesired protein formation.

One method for inhibiting specific gene expression is with the use of oligonucleotides, especially oligonucleotides which are complementary to a specific target messenger RNA (mRNA) sequence.

Transcription factors interact with double-stranded DNA during regulation of transcription. Oligonucleotides can serve as competitive inhibitors of transcription factors to modulate the action of transcription factors. Several recent reports describe such interactions (see Bielinska, A., et. al., Science, 1990, 250, 997-1000; and Wu, H., et. al., Gene, 1990, 89, 203-209).

In addition to functioning as both indirect and direct regulators of proteins, oligonucleotides have also found use in diagnostic tests. Such diagnostic tests can be performed using biological fluids, tissues, intact cells or isolated cellular components. As with gene expression inhibition, diagnostic applications utilize the ability of oligonucleotides to hybridize with a complementary strand of nucleic acid. Hybridization is the sequence specific hydrogen bonding of oligonucleotides, via Watson-Crick and/or Hoogsteen base pairs, to RNA or DNA. The bases of such base pairs are said to be complementary to one another.

Oligonucleotides are also widely used as research reagents. They are useful for understanding the function of many other biological molecules as well as in the preparation of other biological molecules. For example, the use of oligonucleotides as primers in polymerase chain reactions (PCR) has given rise to an expanding commercial industry. PCR has become a mainstay of commercial and research laboratories, and applications of PCR have multiplied. For example, PCR technology is used in the fields of forensics, paleontology, evolutionary studies and genetic counseling. Commercialization has led to the development of kits which assist non-molecular biology-trained personnel in applying PCR. Oligonucleotides, both natural and synthetic, are employed as primers in PCR technology.

Laboratory uses of oligonucleotides are described generally in laboratory manuals such as Molecular Cloning, A Laboratory Manual, Second Ed., J. Sambrook, et al., Eds., Cold Spring Harbor Laboratory Press, 1989; and Current Protocols In Molecular Biology, F. M. Ausubel, et al., Eds., Current Publications, 1993. Such uses include Synthetic Oligonucleotide Probes, Screening Expression Libraries with Antibodies and Oligonucleotides, DNA Sequencing, In Vitro Amplification of DNA by the Polymerase Chain Reaction and Site-directed Mutagenesis of Cloned DNA (see Book 2 of Molecular Cloning, A Laboratory Manual, ibid.) and DNA-Protein Interactions and The Polymerase Chain Reaction (see Vol. 2 of Current Protocols In Molecular Biology, ibid).

Oligonucleotides can be custom-synthesized for a desired use. Thus a number of chemical modifications have been introduced into oligonucleotides to increase their usefulness in diagnostics, as research reagents and as therapeutic entities. Such modifications include those designed to increase binding to a target strand (i.e. increase their melting temperatures, (Tm)); to assist in identification of the oligonucleotide or an oligonucleotide-target complex; to increase cell penetration; to stabilize against nucleases and other enzymes that degrade or interfere with the structure or activity of the oligonucleotides; to provide a mode of disruption (terminating event) once sequence-specifically bound to a target; and to improve the pharmacokinetic properties of the oligonucleotides.

Thus, it is of increasing value to prepare oligonucleotides and other phosphorus-linked oligomers for use in basic research or for diagnostic or therapeutic applications. Consequently, and in view of the considerable expense and time required for synthesis of specific oligonucleotides, there has been a longstanding effort to develop successful methodologies for the preparation of specific oligonucleotides with increased efficiency and product purity.

Synthesis of oligonucleotides can be accomplished using both solution phase and solid phase methods. Oligonucleotide synthesis via solution phase in turn can be accomplished with several coupling mechanisms. However, solution phase chemistry requires purification after each internucleotide coupling, which is labor intensive and time consuming.

The current method of choice for the preparation of naturally occurring oligonucleotides, as well as modified oligonucleotides such as phosphorothioate and phosphorodithioate oligonucleotides, is via solid-phase synthesis wherein an oligonucleotide is prepared on a polymer support (a solid support) such as controlled pore glass (CPG); oxalyl-controlled pore glass (see, e.g., Alul, et al., Nucleic Acids Research 1991, 19, 1527); TENTAGEL Support, (see, e.g., Wright, et al., Tetrahedron Letters 1993, 34, 3373); or POROS, a polystyrene resin available from Perceptive Biosystems.

Solid-phase synthesis relies on sequential addition of nucleotides to one end of a growing oligonucleotide chain. Typically, a first nucleoside (having protecting groups on any exocyclic amine functionalities present) is attached to an appropriate glass bead support and activated phosphite compounds (typically nucleotide phosphoramidites, also bearing appropriate protecting groups) are added stepwise to elongate the growing oligonucleotide. The nucleotide phosphoramidites are reacted with the growing oligonucleotide using “fluidized bed” technology to mix the reagents. The known silica supports suitable for anchoring the oligonucleotide are very fragile and thus cannot be exposed to aggressive mixing. Brill, W. K. D., et al. J. Am. Chem. Soc., 1989, 111, 2321, disclosed a procedure wherein an aryl mercaptan is substituted for the nucleotide phosphoramidite to prepare phosphorodithioate oligonucleotides on glass supports.

Additional methods for solid-phase synthesis may be found in Caruthers U.S. Pat. Nos. 4,415,732; 4,458,066; 4,500,707; 4,668,777; 4,973,679; and 5,132,418; and

Koster U.S. Pat. Nos. 4,725,677 and Re. 34,069.

The preparation of synthetic oligodeoxynucleotides is currently a well-established procedure that is carried out automatically on solid phase using either phosphoramidite (See (a) Caruthers, M. H. Acc. Chem. Res. 1991, 24, 278-284; (b) Beaucage, S. L.; Iyer, R. P. Tetrahedron 1992, 48, 2223-2311) or H-phosphonate chemistry (See (a) Hall, R. H.; Todd, A.; Webb, R. F. J. Chem. Soc. 1957, 3291-3296; (b) Garegg, P. J.; Regberg, T.; Stawinski, J.; Stromberg, R. Chem. Scripta 1985, 25, 280-282; (c) Froehler, B. C.; Matteucci, M. D. Tetrahedron Lett. 1986, 27, 469-472). In both approaches, the synthesis is implemented by stepwise coupling of monomeric nucleoside building blocks to the 5′-terminus of the oligonucleotide chain to be elongated. In the phosphoramidite elongation cycle, the newly formed phosphite triester moiety is subsequently oxidized to give the phosphate triester or sulfurized to the thionophosphate triester. These moieties remain protected until the completion of the synthesis. The phosphate protecting group (or “phosphoryl protecting group”), which is most often the 2-cyanoethyl group (See (a) Sinha, N. D.; Biemat, J.; Köster, H. Tetrahedron Lett. 1983, 24, 5843-5846; and (b) Sinha, N. D.; Biernat, J.; McManus, J.; Köster, H. Nucleic Acids Res. 1984, 12, 4539-4557), is then removed under basic conditions. Thus, the 2-cyanoethyl or other phosphate protecting groups are not removed until the final deprotection of an oligonucleotide.

However, it has been reported that o-methylbenzyl protection can be removed by treatment with a solution of iodine during the normal oxidation protocol (See Caruthers, M. H. Kierzek, R.; Tang, J. Y. In Biophosphates and their Analogues—Synthesis, Structure, Metabolism and Activity; Bruzik, K. S.; and Stec, W. J. Eds.; Esevier: Amsterdam, 1987; pp. 3-21). Thymidine o-methylbenzyl phosphoramidite was shown to couple to the 5′-hydroxy group of phosphate-unprotected, solid support-bound oligodeoxyribonucleotides to afford eicosathymidylate with an average stepwise yield of 96%, which is not optimal for synthesis of phosphodiester and phosphorothioate oligonucleotides.

In addition, it is often desirable to prepare oligonucleotides having a plurality of regions that differ with respect to internucleoside linkage, for example oligonucleotides with mixed backbones that require the successive use of H-phosphonate and phosphoramidite approaches. There is a need for synthetic methodologies for the preparation of such compounds, that provide for the coupling of phosphoramidite synthons to growing oligonucleotide or oligonucleotide analog chains that possess unprotected phosphoryl protecting groups, with high yield. There also is a need for efficient methodologies for the synthesis of oligonucleotides and their analogs that provide for the use of very labile phosphate protecting groups that can be removed under standard automated synthesis conditions. The present invention is directed to these, as well as other, important ends.

### SUMMARY OF THE INVENTION

The present invention provides improved methods for the preparation of oligonucleotides and other phosphorus-linked oligomers. In some preferred embodiments, the present invention provides methods for the preparation of oligonucleotides and phosphorus-linked oligomers wherein monomeric or higher order subunits are added to growing oligomer chains that possess one or more phosphoryl internucleoside linkages that do not bear phosphoryl protecting groups.

Thus, in accordance with some preferred embodiments of the invention, methods are provided comprising reacting a nucleoside phosphoramidite with a support bound oligomer in the presence of a neutralizing agent, said support bound oligomer having at least one unprotected internucleoside linkage selected from the group consisting of phosphate linkages, phosphorothioate linkages, and phosphorodithioate linkages;

wherein said neutralizing agent is:

an aliphatic amine, an aliphatic heterocyclic amine, an aromatic amine, an aromatic heterocyclic amine, a guanidine, or a salt of formula D⁺E⁻ wherein:

D⁺ is a quaternary tetraalkylammonium cation, or a protonated form of an aliphatic amine, an aliphatic heterocyclic amine, an aromatic amine, an aromatic heterocyclic amine, or a guanidine; and

E⁻ is a tetrazolide anion, 4,5-dicyanoimidazolide anion, a substituted or unsubstituted alkylsulfonate anion, a substituted or unsubstituted arylsulfonate anion, tetrafluoroborate anion, hexafluorophosphate anion, or a trihaloacetate anion.

In further preferred embodiments, the present invention provides methods of forming an internucleoside linkage comprising reacting a phosphoramidite of formula:

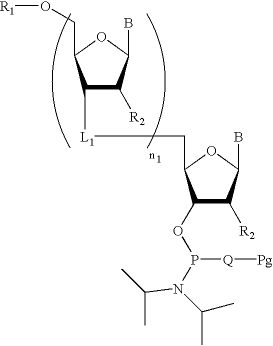

wherein:

L₁ is an internucleoside linkage;

n₁ is 0 to about 100;

R₁ is a hydroxyl protecting group;

R₂ is a 2′-substituent group;

R₄ and R₅ are each independently alkyl having from 1 to about 10 carbon atoms, or R₄ and R₅ taken together with the nitrogen atom to which they are attached form a heterocycle;

B is a nucleobase;

Q is O or S;

Pg is a phosphoryl protecting group;

with a compound of formula:

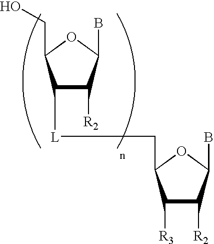

wherein

R₃ is a linker connected to a solid support;

n is from 1 to 100; and

L is an internucleoside linkage of formula:

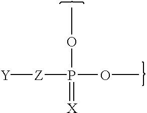

wherein:

Z is O or S;

X is O or S; and

Y is a phosphoryl protecting group or a negative charge;

provided that at least one Y is a negative charge;

wherein said reaction is performed in the presence of a neutralizing agent;

wherein said neutralizing agent is:

an aliphatic amine, an aliphatic heterocyclic amine, an aromatic amine, an aromatic heterocyclic amine, a guanidine, or a salt of formula D⁺E⁻ wherein:

D⁺is a quaternary tetraalkylammonium cation, or a protonated form of an aliphatic amine, an aliphatic heterocyclic amine, an aromatic amine, an aromatic heterocyclic amine, or a guanidine; and

E⁻ is a tetrazolide anion, 4,5-dicyanoimidazolide anion, a substituted or unsubstituted alkylsulfonate anion, a substituted or unsubstituted arylsulfonate anion, tetrafluoroborate anion, hexafluorophosphate anion, or a trihaloacetate anion.

In accordance with further preferred embodiments of the invention, methods are provided comprising the steps of:

(a) providing a solid support having a 5′-O-protected phosphorus-linked oligomer bound thereto, said phosphorus-linked oligomer having at least one phosphoryl internucleoside linkage that does not bear a phosphoryl protecting group;

(b) deprotecting the 5′-hydroxyl of the phosphorus-linked oligomer with a deprotecting reagent;

(c) washing the deprotected phosphorus-linked oligomer on the solid support with a solution containing a neutralizing agent;

(d) reacting the deprotected 5′-hydroxyl with an 5′-protected nucleoside phosphoramidite to produce a phosphite triester linkage therebetween; and

(e) oxidizing or sulfurizing the covalent linkage to form a phosphodiester, phosphorothioate, or phosphorodithioate linkage; and

optionally repeating steps b through e at least once for subsequent couplings of additional nucleoside phosphoramidites;

wherein said neutralizing agent is:

an aliphatic amine, an aliphatic heterocyclic amine, an aromatic amine, an aromatic heterocyclic amine, a guanidine, or a salt of formula D⁺E⁻ wherein:

D⁺ is a quaternary tetraalkylammonium cation, or a protonated form of an aliphatic amine, an aliphatic heterocyclic amine, an aromatic amine, an aromatic heterocyclic amine, or a guanidine; and

E⁻ is a tetrazolide anion, 4,5-dicyanoimidazolide anion, a substituted or unsubstituted alkylsulfonate anion, a substituted or unsubstituted arylsulfonate anion, tetrafluoroborate anion, hexafluorophosphate anion, or a trihaloacetate anion.

In accordance with still further preferred embodiments of the invention, methods are provided comprising the steps of:

(a) providing a solid support having a 5′-O-protected phosphorus-linked oligomer bound thereto, said phosphorus-linked oligomer having at least one phosphoryl internucleoside linkage that does not bear a phosphoryl protecting group;

(b) deprotecting the 5′-hydroxyl of the 5′-O-protected phosphorus-linked oligomer with a deprotecting reagent to form a support bound 5′-deprotected phosphorus-linked oligomer;

(c) optionally washing the 5′-deprotected phosphorus-linked oligomer on the solid support;

(d) contacting the support bound 5′-deprotected phosphorus-linked oligomer with a solution comprising a 5′-protected nucleoside phosphoramidite to produce a phosphite triester linkage therebetween, wherein said solution further comprises a neutralizing agent; and

(e) oxidizing or sulfurizing the phosphite triester linkage to form a phosphodiester, phosphorothioate, or phosphorodithioate linkage; and

optionally repeating steps b through e at least once for subsequent couplings of additional nucleoside phosphoramidites;

wherein said neutralizing agent is:

an aliphatic amine, an aliphatic heterocyclic amine, an aromatic amine, an aromatic heterocyclic amine, a guanidine, or a salt of formula D⁺E⁻ wherein:

D⁺ is a quaternary tetraalkylammonium cation, or a protonated form of an aliphatic amine, an aliphatic heterocyclic amine, an aromatic amine, an aromatic heterocyclic amine, or a guanidine; and

E⁻ is a tetrazolide anion, 4,5-dicyanoimidazolide anion, a substituted or unsubstituted alkylsulfonate anion, a substituted or unsubstituted arylsulfonate anion, tetrafluoroborate anion, hexafluorophosphate anion, or a trihaloacetate anion.

In some preferred embodiments of the forgoing methods, the neutralizing agent is a salt of formula D⁺E⁻. In some preferred embodiments E⁻ is a tetrazolide anion, preferably 1H-tetrazolide anion, 5-methylthio-1H-tetrazolide anion, 5-ethylthio-1H-tetrazolide anion or 1-phenyl-5-thiol-1H-tetrazolide anion, with 1H-tetrazolide anion being most preferred.

In some preferred embodiments, D⁺ is a protonated form of any of an alkyl, alkenyl or alkynyl amine having from one to about 20 carbons, an aliphatic heterocyclic amine, an aromatic heterocyclic amine, or a guanidine.

In some preferred embodiments, the protonated form of an alkyl amine is a protonated form of trimethyl amine, triethyl amine, triisopropyl amine, tributyl amine, triamyl amine, isopropyldimethyl amine, t-butyldimethyl amine, diisopropylethyl amine, or N,N,N′,N′-tetramethyl-1,2-diaminoethane. In further preferred embodiments, D⁺ is a protonated form of an aliphatic heterocyclic amine, which is preferably a protonated form of any of DBU, -methylmorpholine, N-methylpyrrolidine, N-methylpiperidine, N,N′-dimethylpiperazine, N-ethylpyrrolidine, N-ethylpiperidine, N,N′-diethylpiperazine, 1,5-diazabicyclo[4.3.0]non-5-ene, 1,4-diazabicyclo[2.2.2]octane, or 1,5,7-triazabicyclo[4.4.0]dec-5ene.

In further preferred embodiments, D⁺ is a protonated form of an aromatic heterocyclic amine, which is preferably a protonated form of a mono-, di- or trialkyl pyridine that is optionally substituted with an amino group, with a protonated form of any of 2,4,6-collidine, 2,6-lutidine, pyridine, 2-methylpyridine, 2,6-diethylpyridine, 2,6-di(t-butyl)pyridine, 4-methyl-2,6-di(t-butyl)pyridine, or 2,4,6-tri(t-butyl)pyridine being preferred, and with a protonated form of an alkylamino substituted pyridine being more preferred, and with a protonated form of 4-dimethylaminopyridine being even more preferred.

In further preferred embodiments D⁺ is a protonated form of guanidine, preferably tetraalkyl guanidine, with a protonated form of N,N,N′N′-tetramethylguanidine being more preferred. In still further preferred embodiments, D⁺ is a quaternary tetraalkylammonium cation which is preferably a tetramethylammonium, tetraethylammonium, tetrapropylammonium, tetrabutylammonium, trimethyloctylammonium, or triethylbenzylammonium cation.

In further preferred embodiments, E⁻ is 4,5-dicyanoimidazolide anion. In still further preferred embodiments, E⁻ is a substituted or unsubstituted alkylsulfonate anion, with methylsulfonate anion or trifluoromethylsulfonate anion being more preferred.

In some preferred embodiments, E⁻ is a substituted or unsubstituted arylsulfonate anion, with a methylphenylsulfonate anion or a trihalomethylphenylsulfonate anion being more preferred. Preferably, the trihalomethylphenylsulfonate anion is trifluoromethylphenylsulfonate anion.

In further preferred embodiments E⁻ is tetrafluoroborate anion, hexafluorophosphate anion, or a trihaloacetate anion, which is preferably trifluoroacetate anion. In certain preferred embodiments, E⁻ is a tetrazolide anion or substituted or unsubstituted alkylsulfonate anion, and D⁺ is a tetramethylammonium, tetraethylammonium, tetrapropylammonium, tetrabutylammonium, trimethyloctylammonium, or triethylbenzylammonium cation. In further preferred embodiments, E⁻ is trifluoromethanesulfonate anion and D⁺ is a protonated form of N-methylimidazole, N-ethylimidazole, or 1, 2, 4-triazole.

In some more preferred embodiments, D⁺ is a protonated form of trimethyl amine, triethyl amine, triisopropyl amine, tributyl amine, triamyl amine, isopropyldimethyl amine, t-butyldimethyl amine, diisopropylethyl amine, N,N,N′,N′-tetramethyl-1,2-diaminoethane, DBU, N-methylmorpholine, N-methylpyrrolidine, N-methylpiperidine, N,N′-dimethylpiperazine, N-ethylpyrrolidine, N-ethylpiperidine, N,N′-diethylpiperazine, 1,5-diazabicyclo[4.3.0]non-5-ene, 1,4-diazabicyclo[2.2.2]octane, or 1,5,7-triazabicyclo[4.4.0]dec-5ene, 2,4,6-collidine, 2,6-lutidine, pyridine, 2-methylpyridine, 2,6-diethylpyridine, 2,6-di(t-butyl)pyridine, 4-methyl-2,6-di(t-butyl)pyridine, or 2,4,6-tri(t-butyl)pyridine, 4-dimethylaminopyridine, or N,N,N′N′-tetramethylguanidine, or tetramethylammonium, tetraethylammonium, tetrapropylammonium, tetrabutylammonium, trimethyloctylammonium, or triethylbenzylammonium cation; and

E⁻ is 1H-tetrazolide anion, 4,5-dicyanoimidazolide anion, methylsulfonate anion, trifluoromethylsulfonate anion, methylphenylsulfonate anion, trifluoromethylphenylsulfonate anion, tetrafluoroborate anion, hexafluorophosphate anion, or trifluoroacetate anion.

In some further preferred embodiments of the methods of the invention, Q is O; Z is O; Pg is β-cyanoethyl, methyl, (N-methyl-N-benzoylamino)ethyl, (N-ethyl-N-benzoylamino)ethyl, 2-[N-methyl-N-(4-methoxybenzoyl)amino]ethyl, 2-(N-isopropyl-N-benzoylamino)ethyl, 2-[N-ethyl-N-(4-methoxybenzoyl)amino]ethyl, 2-[N-isopropyl-N-(4-methoxybenzoyl)amino]ethyl, 2-[N-methyl-N-(4-dimethylaminobenzoyl)amino]ethyl, 2-[N-ethyl-N-(4-dimethylaminobenzoyl)amino]ethyl, 2-[N-isopropyl-N-(4-dimethylaminobenzoyl)amino]ethyl, 2-(thionobenzoylamino)ethyl, 3-(thionobenzoyl-amino)propyl, 2-(N-phenylthiocarbamoylamino)ethyl, 2-[(1-naphthyl)carbamoyloxy]ethyl, diphenylsilylethyl, δ-cyanobutenyl, cyanop-xylyl, methyl-N-trifluoroacetyl ethyl or acetoxy phenoxy ethyl; and Y is β-cyanoethyl, allyl, methyl, (N-methyl-N-benzoylamino)ethyl, (N-ethyl-N-benzoylamino)ethyl, 2-[N-methyl-N-(4-methoxybenzoyl)-amino]ethyl, 2-(N-isopropyl-N-benzoylamino)ethyl, 2-[N-ethyl-N-(4-methoxybenzoyl)-amino]ethyl, 2-[N-isopropyl-N-(4-methoxybenzoyl)amino]ethyl, 2-[N-methyl-N-(4-dimethylaminobenzoyl)amino]ethyl, 2-[N-ethyl-N-(4-dimethylaminobenzoyl)amino]ethyl, 2-[N-isopropyl-N-(4-dimethylaminobenzoyl)amino]ethyl, 2-(thionobenzoylamino)ethyl, 3-(thionobenzoylamino)propyl, 2-(N-phenylthiocarbamoylamino)ethyl, 2-[(1-naphthyl)carbamoyloxy]ethyl, diphenylsilylethyl, δ-cyanobutenyl, cyanop-xylyl, methyl-N-trifluoroacetyl ethyl, acetoxy phenoxy ethyl, or a negative charge.

In further more preferred embodiments, the neutralizing agent is a salt of formula D⁺E⁻ where E⁻ is a tetrazolide anion which is preferably 1H-tetrazolide anion; D⁺ is a protonated form of dimethylaminopyridine; Pg is β-cyanoethyl, diphenylsilylethyl, δ-cyanobutenyl, cyano p-xylyl, methyl-N-trifluoroacetyl ethyl or acetoxy phenoxy ethyl; and Y is β-cyanoethyl, allyl, diphenylsilylethyl, δ-cyanobutenyl, cyano p-xylyl, methyl-N-trifluoroacetyl ethyl, acetoxy phenoxy ethyl or a negative charge.

In accordance with further preferred embodiments, the present invention provides compositions comprising a 5′-protected nucleoside phosphoramidite and a salt of formula D⁺E⁻ wherein:

D⁺ is a quaternary tetraalkylammonium cation, or a protonated form of an aliphatic amine, an aliphatic heterocyclic amine, an aromatic amine, an aromatic heterocyclic amine, or a guanidine; and

E⁻ is a tetrazolide anion, 4,5-dicyanoimidazolide anion, a substituted or unsubstituted alkylsulfonate anion, a substituted or unsubstituted arylsulfonate anion, tetrafluoroborate anion, hexafluorophosphate anion, or a trihaloacetate anion.

In some preferred embodiments of the compositions of the invention, E⁻ is a tetrazolide anion which is preferably 1H-tetrazolide anion; and D⁺ is a protonated form of a mono-, di- or trialkyl pyridine that is optionally substituted with an amino group, which is preferably a protonated form of dimethylaminopyridine.

In accordance with further preferred embodiments, the present invention provides compositions as described above, and further comprising a solid support having an oligonucleotide or oligonucleotide analog bound thereto, said oligonucleotide or oligonucleotide analog having at least one phosphoryl internucleoside linkage that does not bear a phosphoryl protecting group.

### BRIEF DESCRIPTION OF THE DRAWINGS

FIG. 1 shows the total yields of the 5′-segments of 16a (squares) and 18a (circles) as a function of pK_{BH}+ (MeCN) values of protonated nitrogen bases. (a) Standard cycle (shown as a horizontal grid); (b) Py; (c) Lut; (d) Col; (e) NMM; (f) DIPEA; (g) TEA.

FIG. 2 shows the total yields of the 5′-segments of 16a (squares) and 18a (circles) as a function of pK_{BH}+ (MeCN) values of protonated nitrogen bases. (a) Standard cycle (shown as a horizontal grid); (b) Py; (c) Lut; (d) Col; (e) NMM-Tet; (f) DMAP-Tet; (g) DIPEA-Tet; (h) TEA-Tet; (I) TBD-Tet; (k) DBU-Tet.

FIG. 3 shows the total yields of the 5′-segments of 16b (▪) and 18b (●) as a function of pK_{BH}+ (MeCN) values of protonated nitrogen bases. (a) Standard cycle (shown as a horizontal grid); (b) Py; (c) Lut; (d) Col; (e) NMM-Tet; (f) DMAP-Tet; (g) DIPEA-Tet; (h) TEA-Tet; (I) TBD-Tet; (k) DBU-Tet.

FIG. 4 shows the ³¹P NMR Spectrum of 3a in gel phase (CD₃CN as a liquid phase).

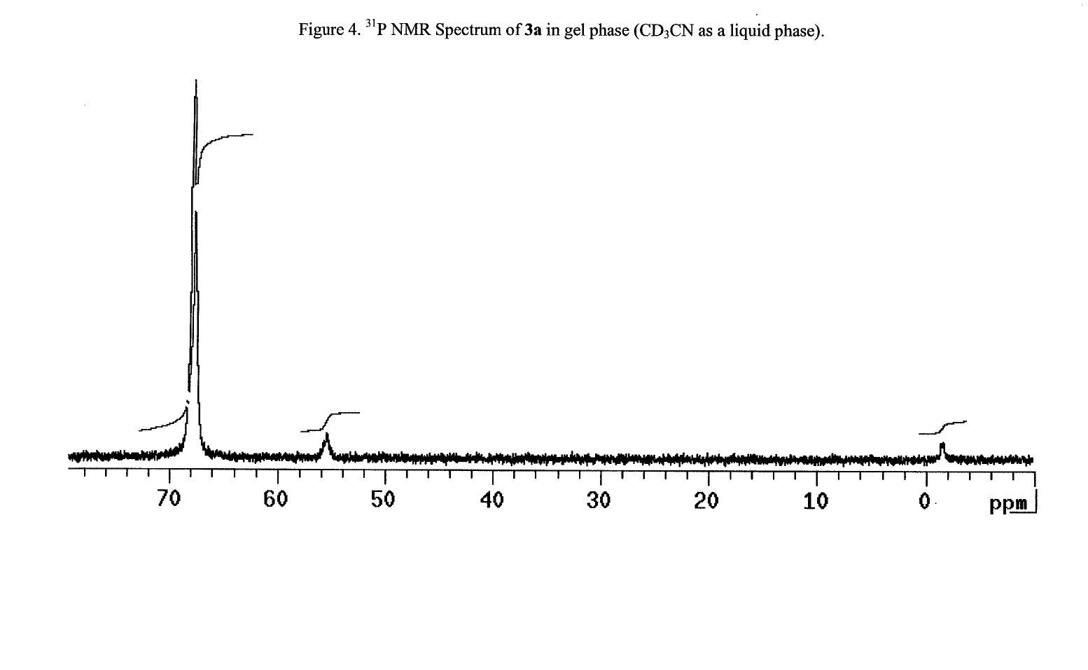

FIG. 5 shows the ³¹P NMR Spectrum of 4a in Gel Phase (IM Piperidine in CD₃CN as a liquid phase).

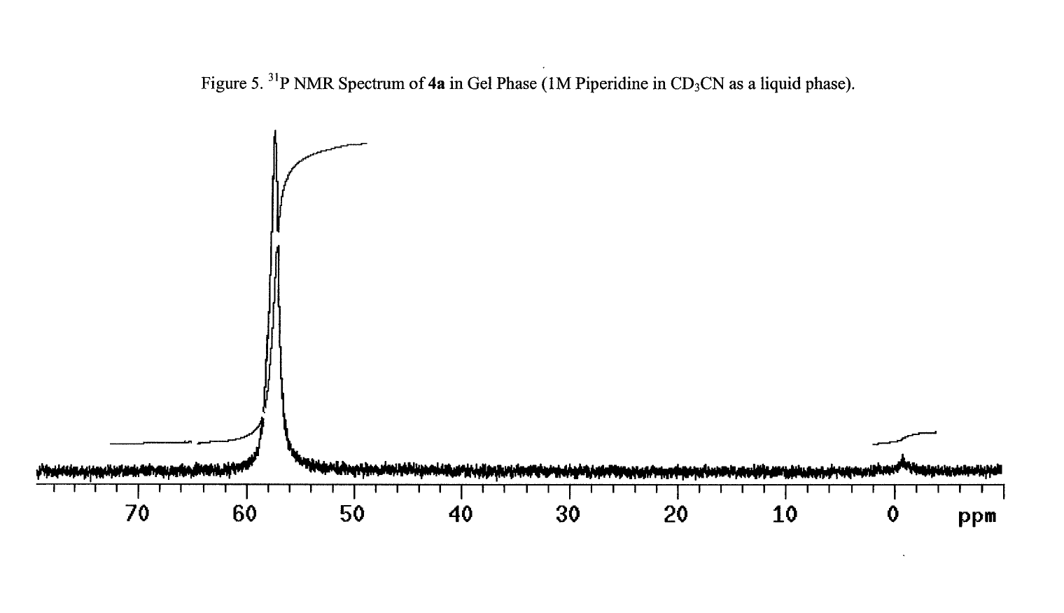

FIG. 6 shows the ³¹P NMR Spectrum of 6a in Gel Phase (5% Pyridine in CD₃CN as a liquid phase).

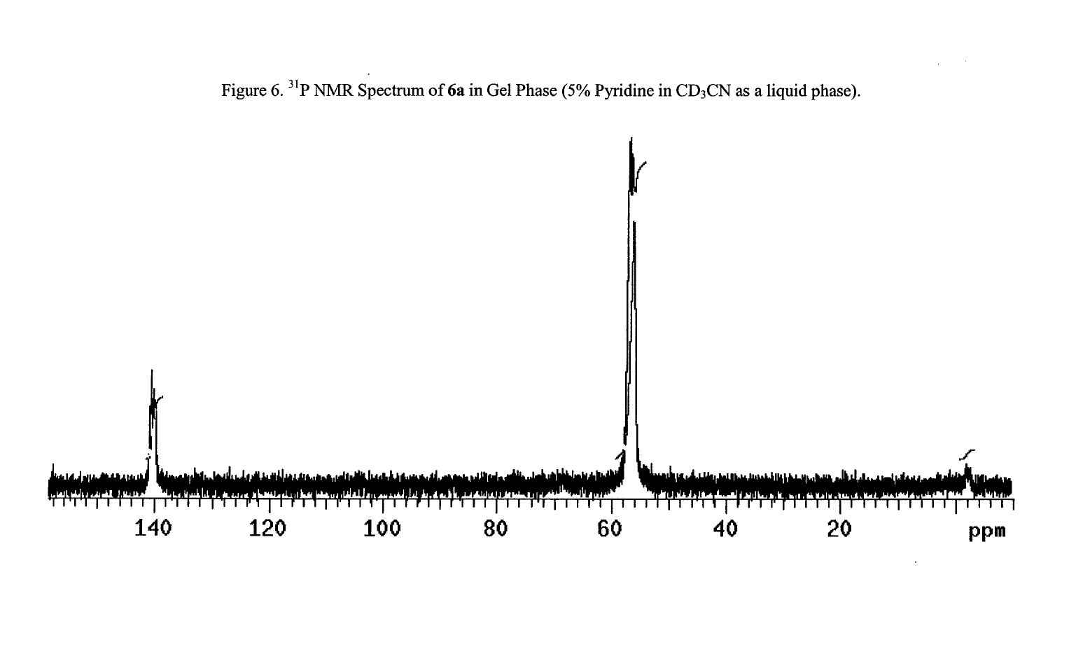

FIG. 7 shows the ³¹P NMR Spectrum of 7a in Gel Phase (5% Pyridine in CD₃CN as a liquid phase).

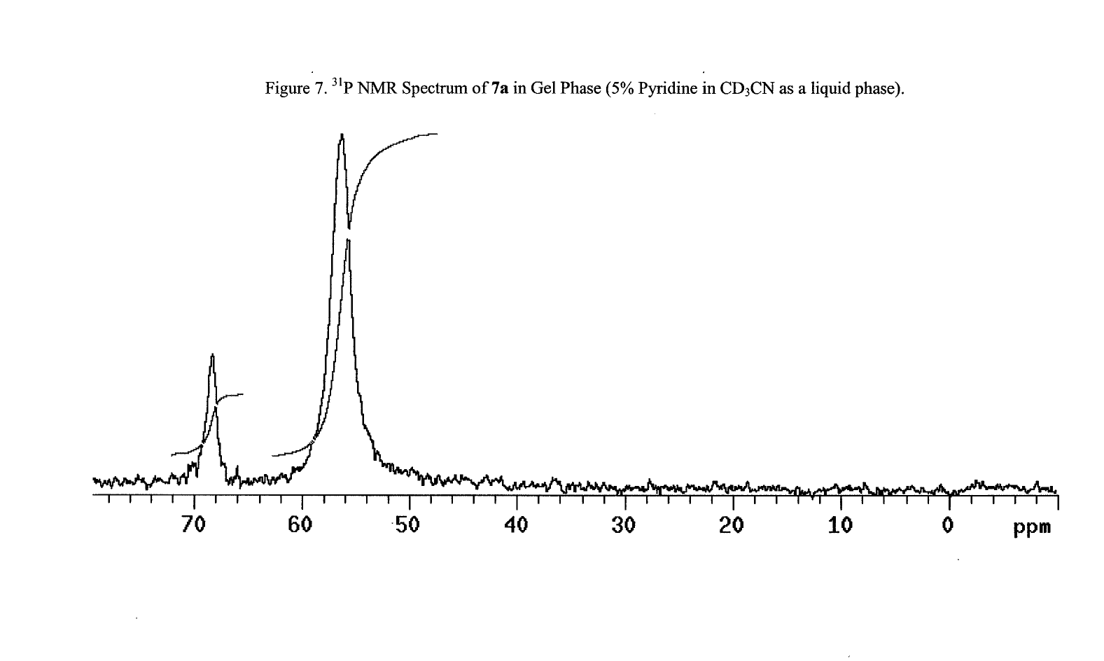

FIG. 8 shows the ³¹P NMR Spectrum of 8a in Gel Phase (5% Pyridine in CD₃CN as a liquid phase).

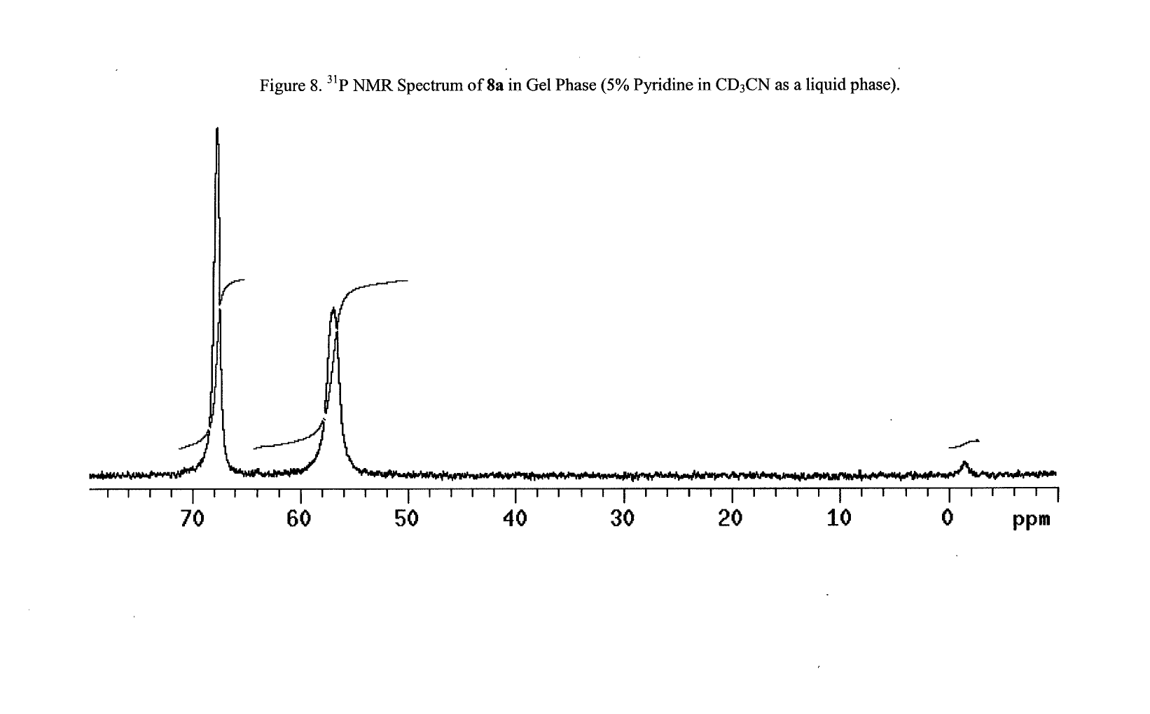

FIG. 9 shows the Reverse Phase HPLC Profile for Oligonucleotide 9a Obtained Using the Standard Cycle (Crude Deprotection Mixture).

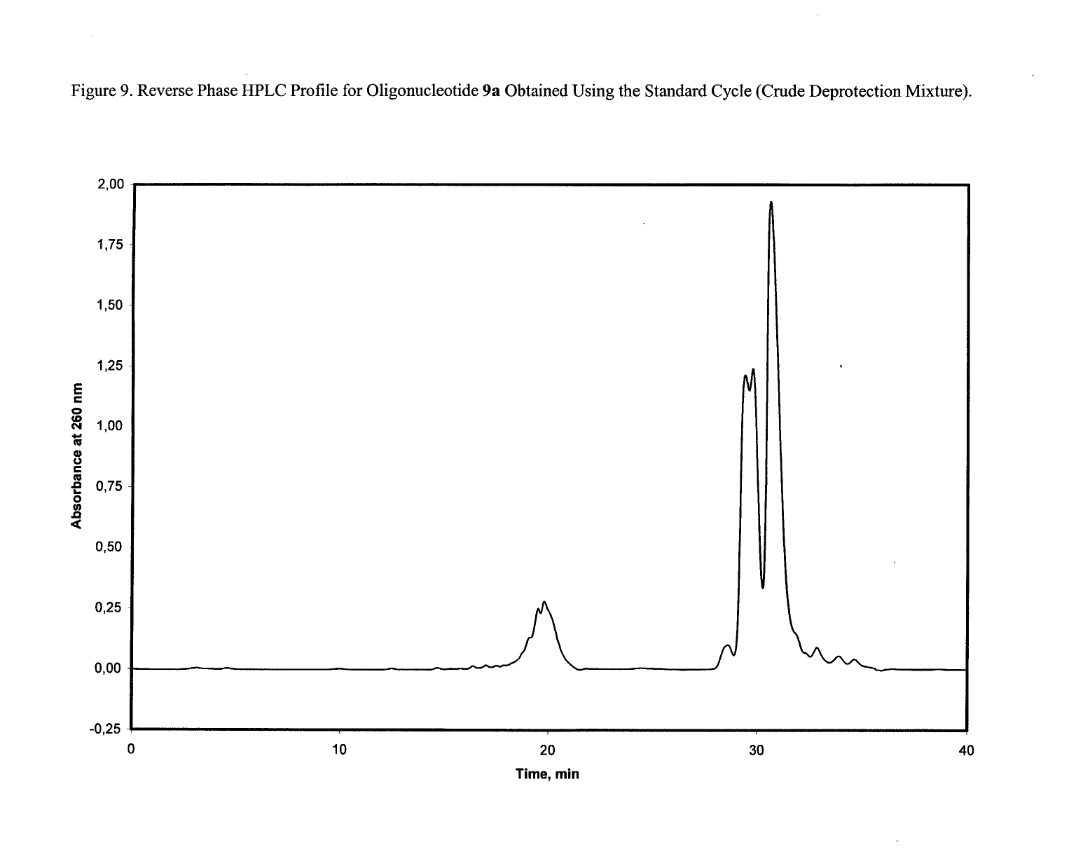

FIG. 10 shows the ³¹P NMR Spectrum of 4b in Gel Phase (1M Piperidine in CD₃CN as a liquid phase).

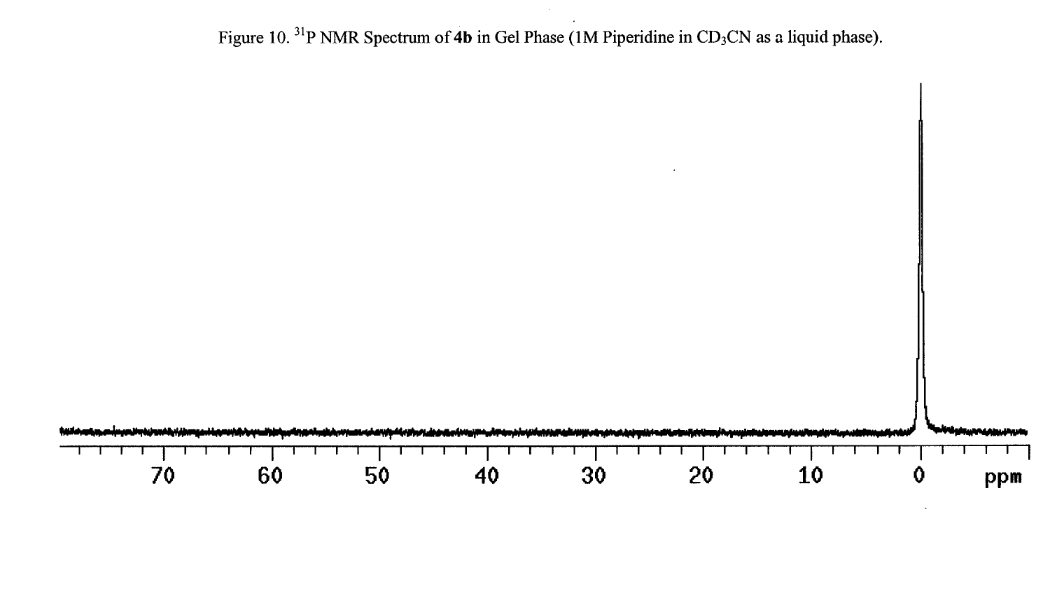

FIG. 11 shows the ³¹P NMR Spectrum of 8b in Gel Phase (5% Pyridine in CD₃CN as a liquid phase).

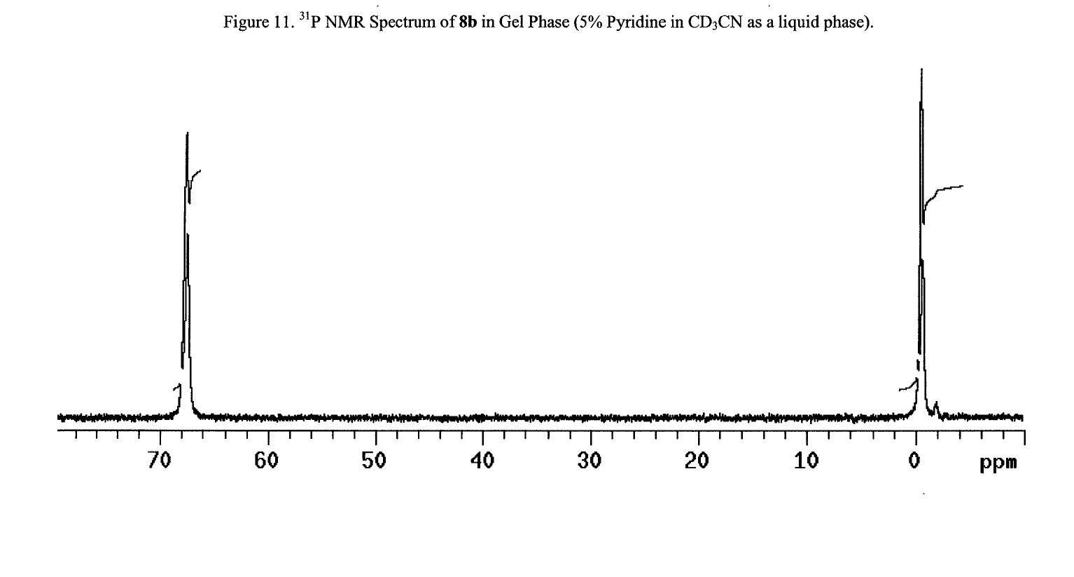

FIG. 12 shows the Reverse Phase HPLC Profile for Oligonucleotide 9b Obtained Using the Standard Cycle (Crude Deprotection Mixture).

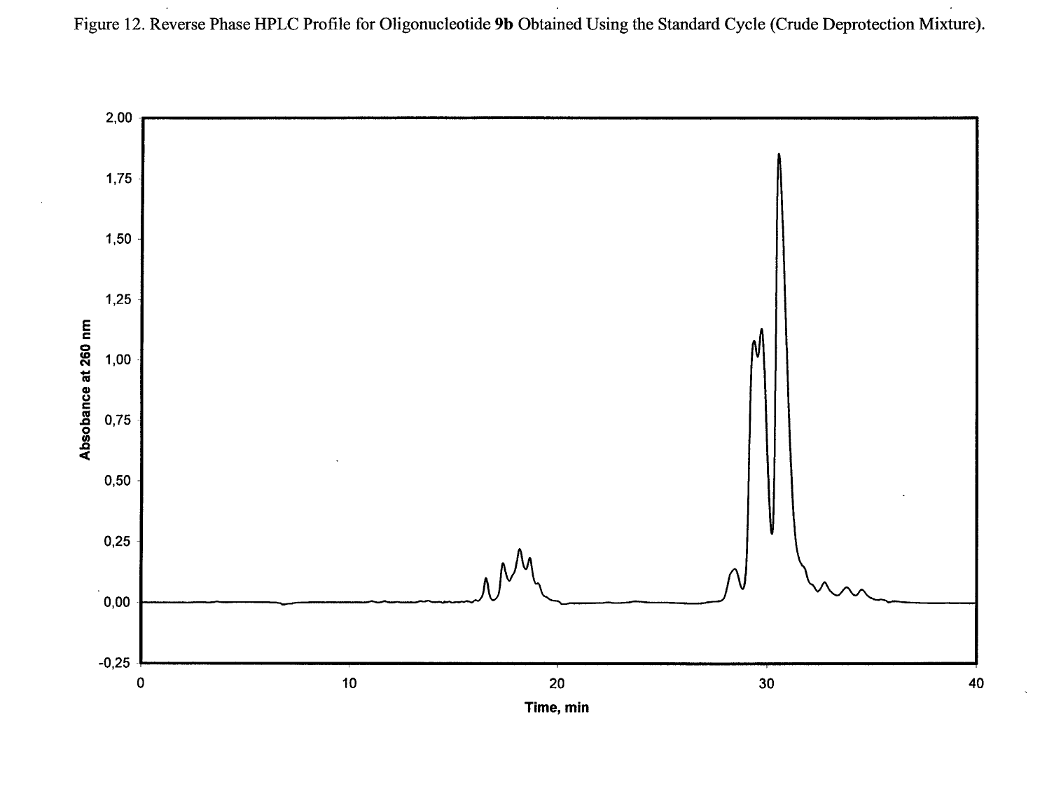

FIG. 13 shows the Reverse Phase HPLC Profile for Oligonucleotide 16a Obtained Using the Standard Cycle (Crude Deprotection Mixture).

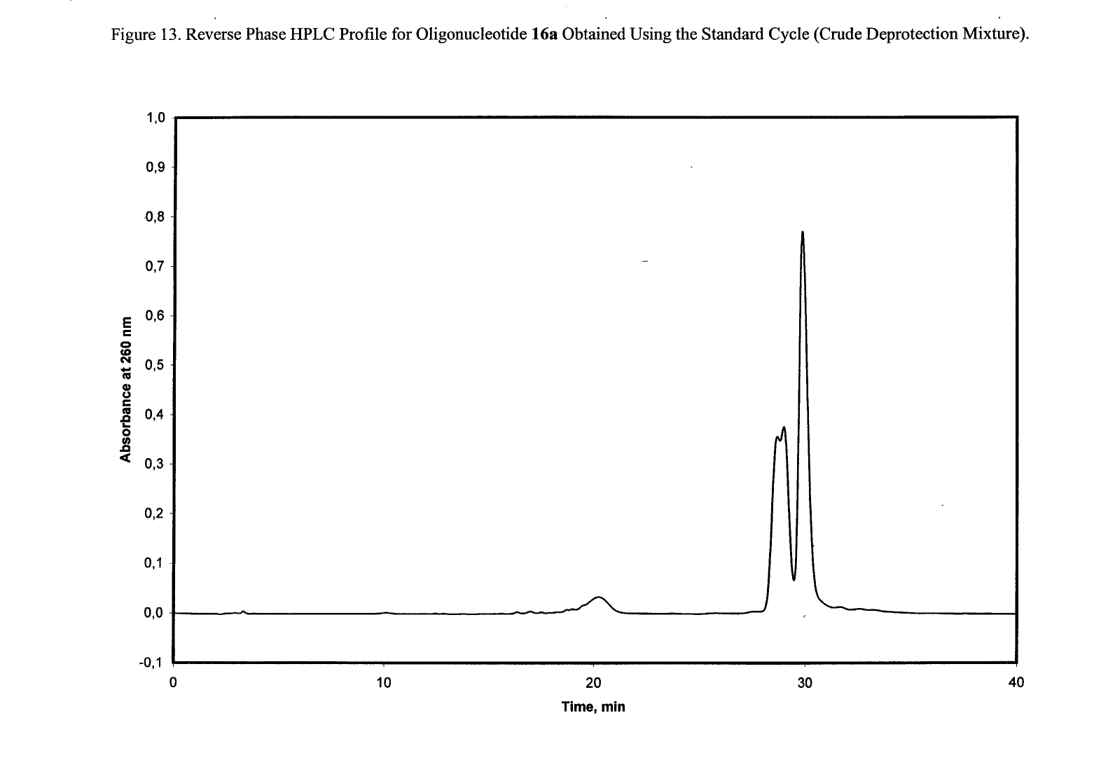

FIG. 14 shows the Reverse Phase HPLC Profile for Oligonucleotide 18a Obtained Using the Standard Cycle (Crude Deprotection Mixture).

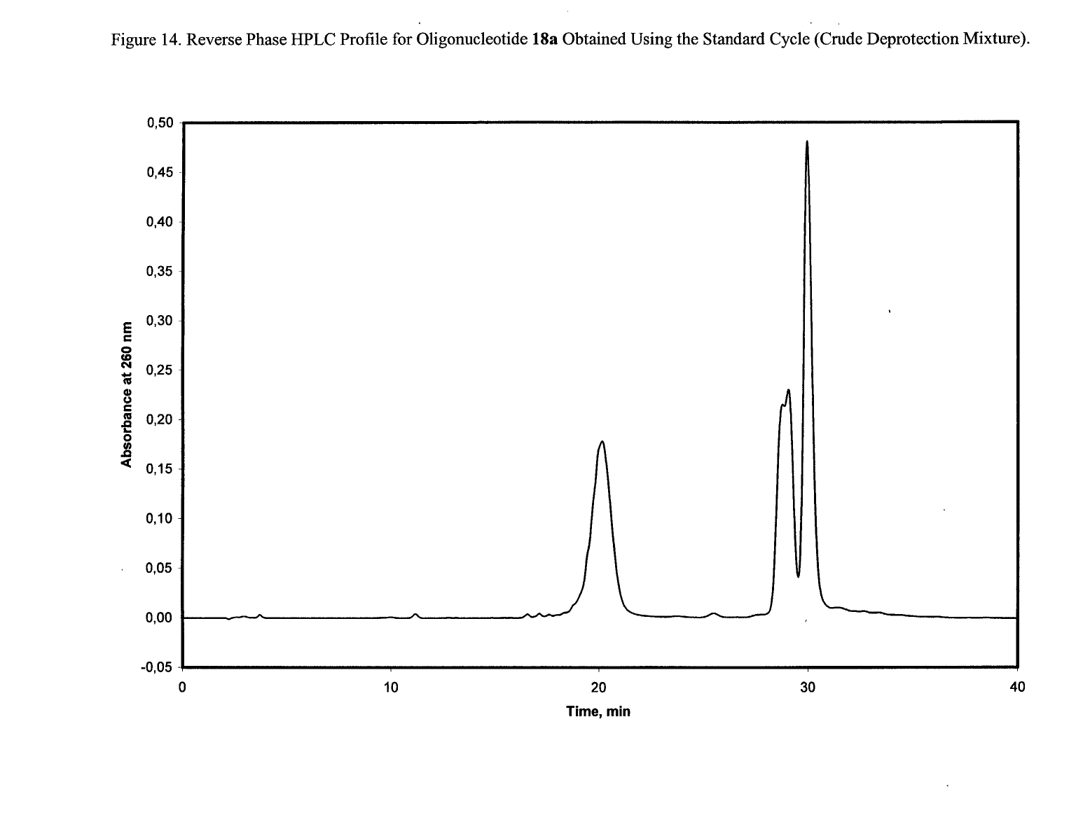

FIG. 15 shows the Reverse Phase HPLC Profile for Oligonucleotide 16b Obtained Using the Standard Cycle (Crude Deprotection Mixture).

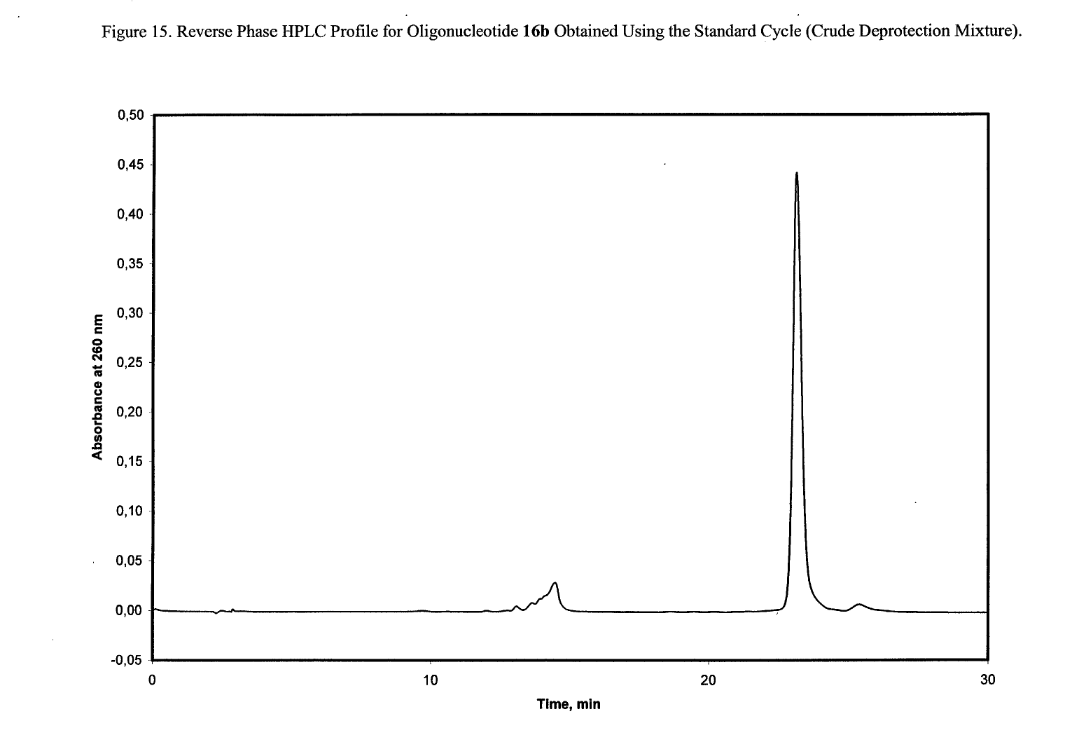

FIG. 16 shows the Reverse Phase HPLC Profile for Oligonucleotide 18b Obtained Using the Standard Cycle (Crude Deprotection Mixture).

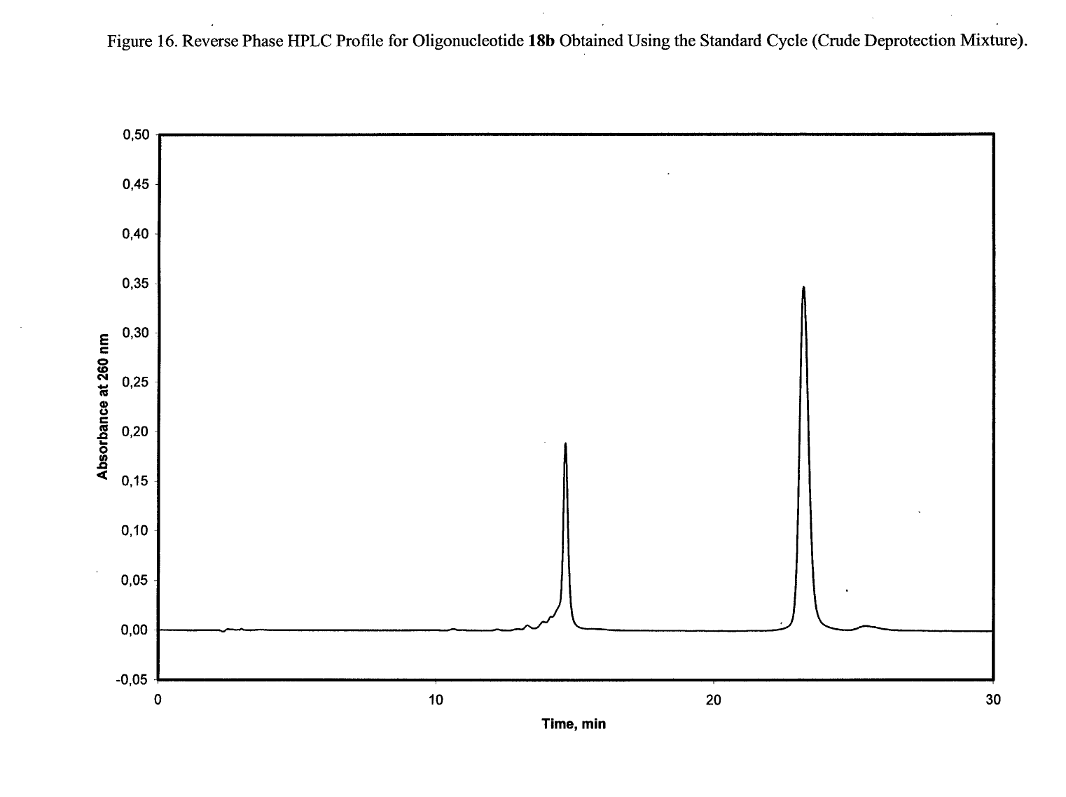

FIG. 17 shows the Reverse Phase HPLC Profile for Oligonucleotide 16a Obtained Using the Optimized Cycle (Crude Deprotection Mixture).

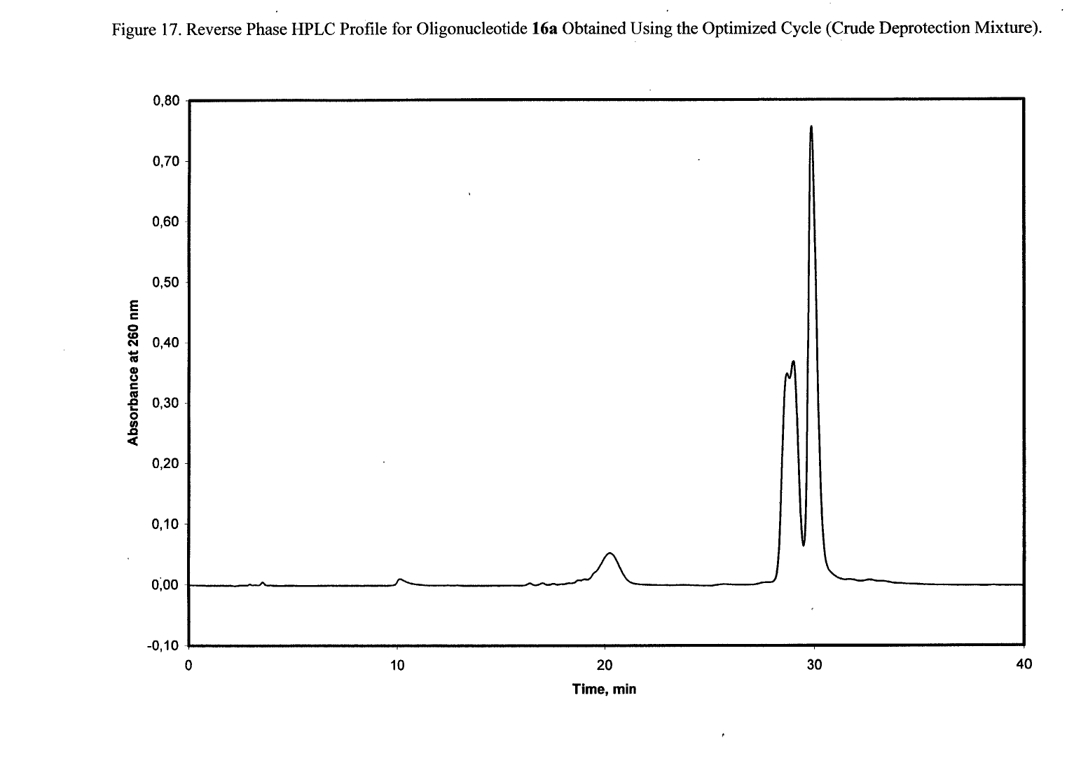

FIG. 18 shows the Reverse Phase HPLC Profile for Oligonucleotide 18a Obtained Using the Optimized Cycle (Crude Deprotection Mixture).

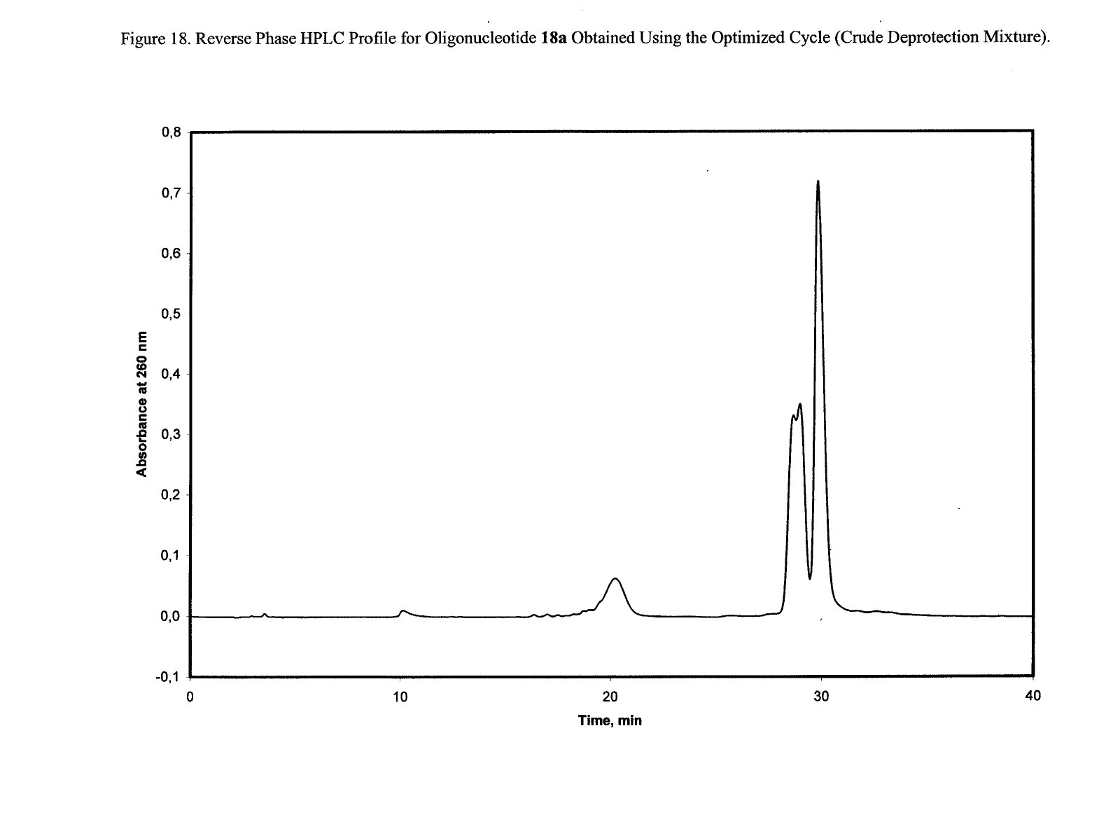

FIG. 19 shows the Reverse Phase HPLC Profile for Oligonucleotide 16b Obtained Using the Optimized Cycle (Crude Deprotection Mixture).

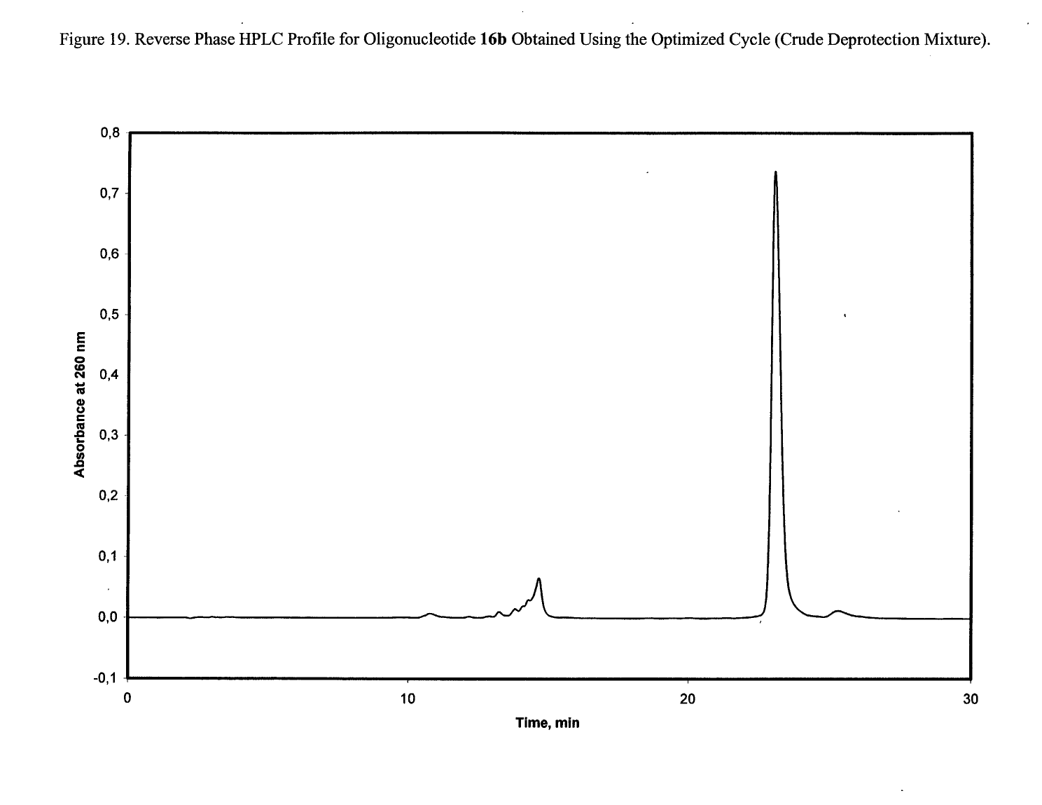

FIG. 20 shows the Reverse Phase HPLC Profile for Oligonucleotide 18b Obtained Using the Optimized Cycle (Crude Deprotection Mixture).

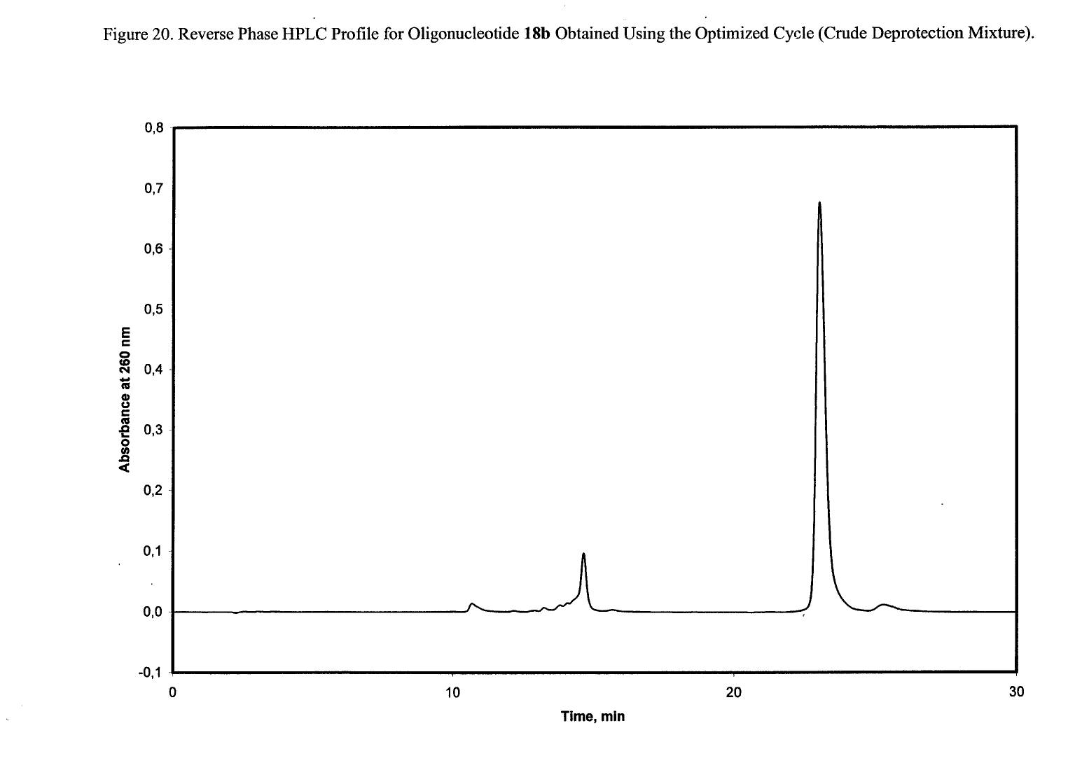

FIG. 21 shows the Reverse Phase HPLC Profile for Oligonucleotide 32a Obtained Using the Optimized Cycle (Crude Deprotection Mixture).

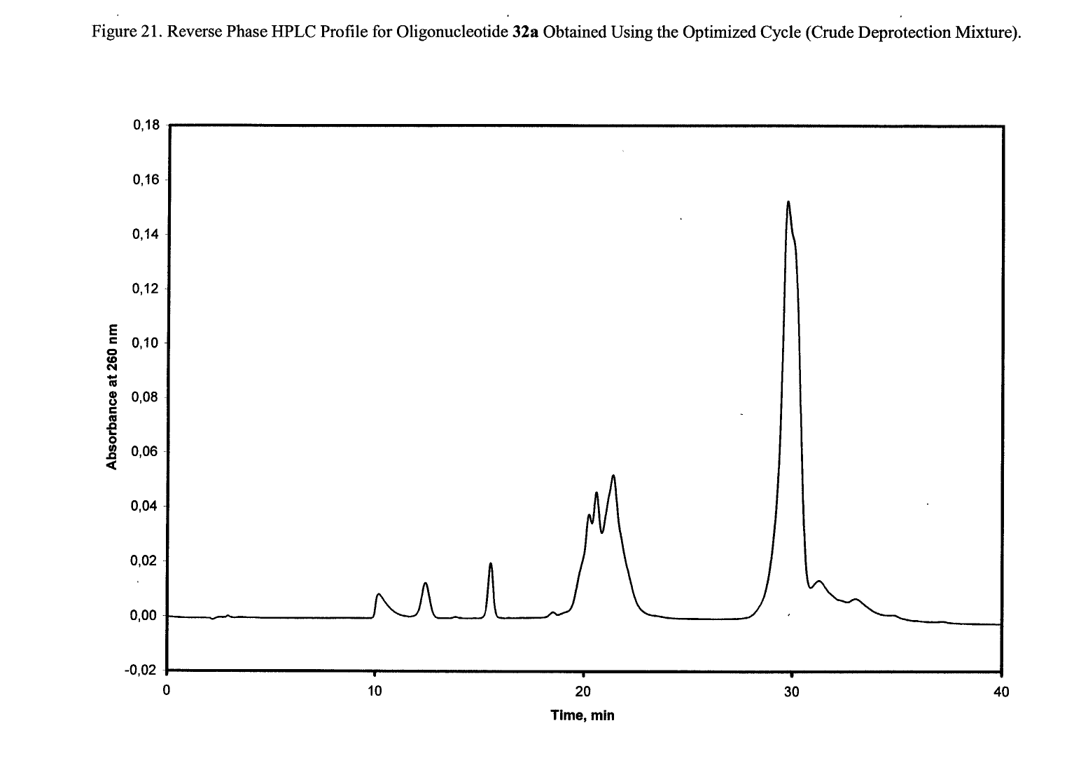

FIG. 22 shows the Reverse Phase HPLC Profile for Oligonucleotide 32b Obtained Using the Optimized Cycle (Crude Deprotection Mixture).

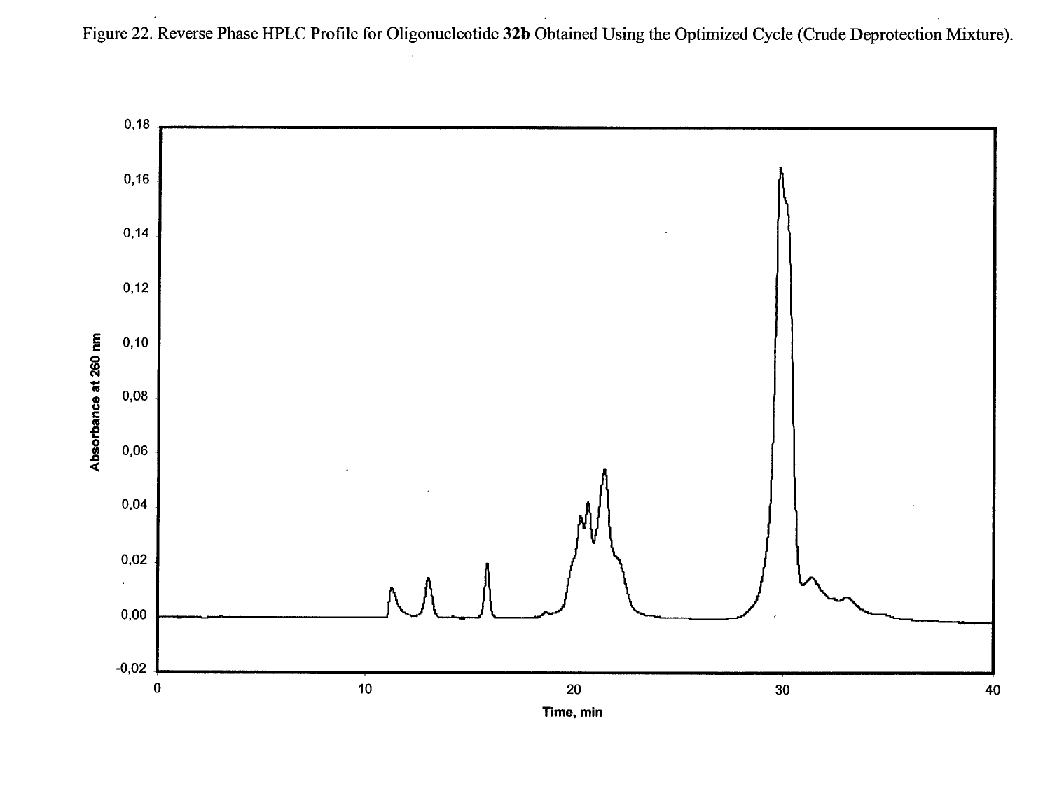

### DETAILED DESCRIPTION

In some preferred embodiments, the present invention provides methods and compositions for the preparation of phosphorus-linked oligomers wherein monomeric or higher order subunits are added to growing oligomer chains that possess one or more phosphoryl internucleoside linkages (for example, phosphodiester, phosphorothioate, phosphorodithioate or H-phosphonate linkages) that do not bear phosphoryl protecting groups. The methods of the invention provide stepwise yields of oligomer that are higher than those obtained in the absence of the neutralizing agents of the invention, particularly when the support bound oligomer to be extended has multiple unprotected phosphoryl internucleoside linkages.

In accordance with some preferred embodiments of the invention, the efficiency of the coupling reaction between phosphoramidite and support-bound oligomer containing at least one unprotected phosphoryl internucleoside linkage is raised to, or exceeds, the commonly accepted levels for synthesis of oliogomers that include, for example, phosphodiester linkages, phosphorothioate linkages, or mixtures thereof.

In some further preferred embodiments of the methods of the invention, an oligonucleotide synthon having a 3′-phosphoramidite group, and at least one unprotected phosphoryl internucleoside linkage, is coupled to a support-bound monomeric or higher order oligomer having an unprotected 3′-hydroxyl, and, optionally, one or more unprotected phosphoryl internucleoside linkages, in the presence of a neutralizing agent as described herein. Thus, in accordance with the methods of the present invention, the unprotected phosphoryl internucleoside linkage or linkages can reside in the phosphoramidite synthon, the support bound oligomer, or both.

In the context of the present invention, the term “phosphorus-linked oligomer” refers to a plurality (i.e., greater than two) of joined nucleobase-bearing monomeric subunits that are connected by linking groups, wherein at least one of the linking groups contains a phosphorus atom. Examples of phosphorus-linked oligomers include those that contain one or more phosphodiester, phosphotriester, phosphorothioate, phosphorodithioate, and H-phosphonate linkages, although phosphorus-linked oligomers may also contain one or more non-phosphorus containing linkages between monomeric subunits. As use herein, the term “internucleoside linkage” is intended to mean a linkage between hydroxyl groups of two nucleosidic sugars (e.g., ribose, substituted ribose, or analogs thereof).

In accordance with some preferred embodiments of the invention, a support bound dimer or oligomer having at least one unprotected phosphorus-containing internucleoside linkage (i.e., at least one unprotected phosphoryl linkage) is reacted with a nucleoside phosphoramidite in the presence of a neutralizing agent to produce an internucleoside linkage in high yield. In accordance with some preferred methods of the invention, such support bound oligomer can have one or more types of phosphorus containing linkages, each of which may be protected (i.e., bear a phosphoryl protecting group) or be unprotected (i.e., lack such a phosphoryl protecting group). Examples of such types of phosphorus containing internucleoside linkages include phosphate linkages, phosphorothioate linkages, phosphorodithioate linkages, and H-phosphonate linkages.

In some preferred embodiments of the methods of the invention, the reaction between the nucleoside phosphoramidite and the support bound oligomer is performed in the presence of a neutralizing agent that is an aliphatic amine, an aliphatic heterocyclic amine, an aromatic amine, an aromatic heterocyclic amine, a guanidine, or a salt of formula D⁺E⁻ wherein:

D⁺ is a quaternary tetraalkylammonium cation, or a protonated form of an aliphatic amine, an aliphatic heterocyclic amine, an aromatic amine, an aromatic heterocyclic amine, or a guanidine; and

E⁻ is a tetrazolide anion, 4,5-dicyanoimidazolide anion, a substituted or unsubstituted alkylsulfonate anion, a substituted or unsubstituted arylsulfonate anion, tetrafluoroborate anion, hexafluorophosphate anion, or a trihaloacetate anion.

The neutralizing agent can be applied in a washing step, prior to coupling of a phosphoramidite, or can be included in the solution applied in the coupling step, along with the nucleoside phosphoramidite to be coupled. Thus, the invention provides for methods for oligonuicleotide synthesis wherein a washing step is employed wherein the washing solution contains a neutralizing agent of the invention. Also provided by the present invention are methods for oligonuicleotide synthesis wherein a neutralizing agent as described herein is included within the solution containing nucleoside or higher order phosphoramidite to be coupled to a growing oligomer chain.

As used herein, the term “a tetrazole” is intended to include 1H-tetrazole and substituted 1H-tetrazoles. Substituted 1H-tetrazoles include lower alkyl substituted (i.e., C₁-C₆)-1H-tetrazoles, lower alkylthio substituted 1H-tetrazoles for example 5-methylthio-1H-tetrazole and 5-ethylthio-1H-tetrazole, and tetrazoles having both thiol and lower alkyl or aryl substituents, for example l-phenyl-5-thiol-1H-tetrazole. Also included in the definition of “a tetrazole” are nitrotetrazoles and N,N-diisopropylaminohydrotetrazolide. As used herein the term “a tetrazolide anion” is intended to mean the corresponding anionic form of a tetrazole, after removal of a proton.

As used herein, the term “tetrazolide” is intended to mean the salt that results from a tetrazole, preferably a 1H-tetrazole, and a base. In some preferred embodiments, the support bound oligomer chain that is desired to be elongated can have one, two, or a plurality of unprotected phosphorus-containing linkages. Thus, in some preferred embodiments, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100% of the phosphorus containing linkages of the support bound oligomer can be unprotected.

In addition to containing at least one unprotected phosphorus-containing internucleoside linkage, the support bound oligomer can contain one or more monomeric units that are linked by protected phosphorus-containing internucleoside linkages. In addition, or alternatively, in some preferred embodiments the support bound oligomer can contain one or more monomeric units that are linked by non-phosphorus containing internucleoside linkages. Examples of such non-phosphorus internucleoside linkages are the methylene-methylimmino linkage (See for example Vasseur, J. J., Debart, F., Sanghvi, Y. S., and Cook, P. D.; 1992, J. Am. Chem. Soc. 114, 4006-7), and the morpholino linkage (See Summerton, J. (1999) Biochim. Biophys. Acta 1489, 141-158). In some preferred embodiments, the support bound oligomer contains two or more regions which have different internucleoside linkages. Thus, in some preferred embodiments, the methods of the invention are employed to prepare such “chimeric” oligomers by coupling protected phosphoramidite monomers or higher order phosphoramidite synthons to an oligomer chain having one or more regions of unprotected phosphate, phosphorothioate, phosphorodithioate or H-phosphonate internucleoside linkages.

In other preferred embodiments, the support bound oligomer can contain one regions that do not possess sugar backbones. One example of such a non-sugar backbone is the peptide nucleic acid (PNA). See Nielsen, P. E., Egholm, M., Berg, R. H., and Buchardt, O. 1991, Science 254,1497-500; Egholm, M., Buchardt, O., Nielsen, P. E., and Berg, R. H. 1992 J Am. Chem. Soc. 114,1895-7; and Corey, D. R. 1997 in Trends Biotechnol. pp 224-229. Thus, in further preferred embodiments, the methods of the invention are employed to prepare chimeric oligomers by coupling protected phosphoramidite monomers on to an oligomer chain having at least one unprotected phosphoryl linkage and one or more regions of non-sugar backbone. Also provided in accordance with preferred embodiments of the invention are compositions comprising a 5′-protected nucleoside phosphoramidite or higher order phosphoramidite synthon and a salt of formula D⁺E⁻ wherein D⁺ is a quaternary tetraalkylammonium cation, or a protonated form of an aliphatic amine, an aliphatic heterocyclic amine, an aromatic amine, an aromatic heterocyclic amine, or a guanidine; and E⁻ is a tetrazolide anion, 4,5-dicyanoimidazolide anion, a substituted or unsubstituted alkylsulfonate anion, a substituted or unsubstituted arylsulfonate anion, tetrafluoroborate anion, hexafluorophosphate anion, or a trihaloacetate anion. In some preferred embodiments, of the compositions of the invention, E⁻ is a tetrazolide anion; and D⁺ is a protonated form of a mono-, di- or trialkyl pyridine that is optionally substituted with an amino group. In some particularly preferred embodiments, E⁻ is 1H-tetrazolide anion; and D⁺ is a protonated form of dimethylaminopyridine.

Further preferred embodiments of the compositions of the invention comprise a neutralizing agent as described herein, and a solid support having phosphorus-linked oligomer bound thereto, said phosphorus-linked oligomer having at least one phosphoryl internucleoside linkage that does not bear a phosphoryl protecting group.

The compositions of the invention are useful in the preparation of phosphorus-linked oligomers as described herein. The compositions of the invention can be used in research reagents and kits. For synthetic applications, kits containing the compositions of the invention can be conveniently used in standard oligonucleotide synthetic regimes, as described herein. The methods of the invention provide stepwise yields of oligomer that are higher than those obtained in the absence of the neutralizing agents of the invention, particularly when the support bound oligomer to be extended has multiple unprotected phosphoryl internucleoside linkages. In some preferred embodiments, the methods of the invention stepwise yields of 96.5%,, 97% 97.2%, 97.4%, 97.6%, 97.8%, 98%, 98.2%, 98.4%, 98.6%, 98.8%, 99%, 99.2%, 99.4%, 99.6% 99.8% or about 100%.

As used herein, the term “aliphatic amine” is intended to mean an aliphatic compound containing at least one amino nitrogen. The term “aliphatic” is intended to mean non-aromatic, although “aliphatic” compounds may have one or more double or triple bonds. Examples of aliphatic amines include alkyl, alkenyl and alkenyl amines, for example trimethyl amine, triethyl amine, triisopropyl amine, and tributyl amine. In more preferred embodiments, the aliphatic amine is a tertiary amine.

The term “aliphatic heterocyclic amine” is intended to denote an aliphatic amine that contains at least one ring that contains at least one heteroatom. The ring heteroatom can be the amino nitrogen, or another hetero (i.e., noncarbon) atom selected from 0, N and S. Examples of aliphatic heterocyclic amines include 1,8 diazabicyclo[5.4.0] undec-7-ene (DBU), morpholine and alkyl-substituted morpholines including N-methylmorpholine (NMM), piperazines, including mono-N-alkyl, di-N-alkyl, mono-N-alk and di-N-aryl piperazines, N-alkyl and N-aryl pyrrolidines, and piperidines, including N-alkyl and N-aryl piperidines.

As used herein, the term “aromatic amine” is intended to mean an aromatic compound that contains at least one amino nitrogen. The term “aromatic heterocyclic amine” is intended to mean an aromatic amine that contains at least one ring that contains at least one heteroatom. The ring heteroatom can be the amino nitrogen, or another hetero (i.e., noncarbon) atom selected from O, N and S. Examples of aromatic heterocyclic amines include pyridine and substituted pyridines including mono-, di- and trialkyl pyridines, alkylamino pyridines, for example dimethylaminopyridine (DMAP), pyrroles including alkyl substituted pyrroles, pyrimidines including alkyl substituted pyrimidines, and imidazoles including alkyl substituted imidazoles.

As used herein, “alkyl” refers to a hydrocarbon containing from 1 to about 20 carbon atoms. Alkyl groups may straight, branched, cyclic, or combinations thereof. Alkyl groups thus include, by way of illustration only, methyl, ethyl, propyl, isopropyl, butyl, isobutyl, cyclopentyl, cyclopentylmethyl, cyclohexyl, cyclohexylmethyl, and the like. Also included within the definition of “alkyl” are fused and/or polycyclic aliphatic cyclic ring systems such as, for example, adamantane. As used herein the term “alkenyl” denotes an alkyl group having at least one carbon-carbon double bond. As used herein the term “alkynyl” denotes an alkyl group having at least one carbon-carbon triple bond.

As used herein, the term “heterocycle” is intended to denote a cyclic aliphatic or aromatic compound that contains from one to three ring hetero atoms selected from 0, N and S. Examples of heterocycles include morpholine, pyran and pyridine.

In some preferred embodiments, the aforementioned aliphatic amines, aliphatic heterocyclic amines, aromatic amines, aromatic heterocyclic amines, guanidines (and protonated forms of the foregoing), tetrazolide anions and quaternary tetraalkylammonium cations can be “substituted”; that is, they can bear one or more further substituent groups. In some preferred embodiments these substituent groups can include halogens, CN, NO₂, lower (i.e., C₁-C₆) alkyl groups, alkoxycarbonyl groups, alkoxy groups, and hydroxy groups.

As used therein the term halogen includes fluorine, chlorine, bromine and iodine.

As used herein the term “aryl” means a group having 5 to about 20 carbon atoms and which contains at least one aromatic ring, such as phenyl, biphenyl and naphthyl. Preferred aryl groups include unsubstituted or substituted phenyl and naphthyl groups, where “substituted” has the meaning described above.

The term “a guanidine” is intended to mean guanidine, and simple momno-, di-, tri- and tetra-N-alkyl substituted guanidines, including N,N, N′N′-tetramethylguanidine.

In accordance with some preferred embodiments of the invention, methods of forming an internucleside linkage are provided comprising reacting a phosphoramidite of formula:

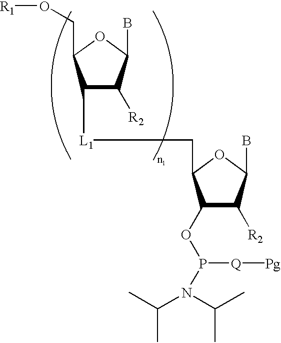

wherein:

L₁ is an internucleoside linkage;

n₁ is 0 to about 100;

R₁ is a hydroxyl protecting group;

R₂ is a 2′-substituent group;

R₄ and R₅ are each independently alkyl having from 1 to about 10 carbon atoms, or R₄ and R₅ taken together with the nitrogen atom to which they are attached form a heterocycle;

B is a nucleobase;

Q is O or S;

Pg is a phosphoryl protecting group;

with a compound of formula:

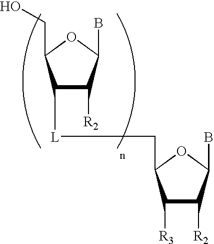

wherein

R₃ is a linker connected to a solid support;

n is from 1 to 100; and

L is an internucleo side linkage of formula:

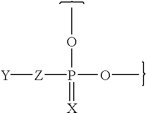

wherein:

Z is O or S;

X is O or S; and

Y is a phosphoryl protecting group or a negative charge;

provided that at least one Y is a negative charge;

wherein said reaction is performed in the presence of a neutralizing agent;

wherein said neutralizing agent is:

an aliphatic amine, an aliphatic heterocyclic amine, an aromatic amine, an aromatic heterocyclic amine, a guanidine, or a salt of formula D⁺E⁻ wherein:

D⁺ is a quaternary tetraalkylammonium cation, or a protonated form of an aliphatic amine, an aliphatic heterocyclic amine, an aromatic amine, an aromatic heterocyclic amine, or a guanidine; and

E⁻ is a tetrazolide anion, 4,5-dicyanoimidazolide anion, a substituted or unsubstituted alkylsulfonate anion, a substituted or unsubstituted arylsulfonate anion, tetrafluoroborate anion, hexafluorophosphate anion, or a trihaloacetate anion.

As can be seen, the methods of the present invention provide for the use of both mononucleoside phosphoramidite synthons, and higher order (i.e., di-, tri-, tetra- or longer) nucleoside phosphoramidite synthons. Such higher order synthons can have phosphate-containing internucleoside linkages which may be protected or unprotected, and also can have non-phosphate-containing internucloside linkages. In addition, or alternatively, such synthons can possess regions of non-sugar containing backbone, for example PNA, and/or regions that differ with respect to sugar substituent, for example regions that differ with respect to 2′-substituent. In some preferred embodiments, the methods of the invention are used to couple an oligonucleotide or analog thereof to a solid-support bound di- or higher order oligomeric species, wherein at least one internucleoside linkage in either the phosphoramidite or the support bound species is an unprotected phosphoryl linkage.

The methods of the invention are amenable to the preparation of oligonucleotides and analogs thereof having a wide variety of modifications, including base modifications, backbone modifications, phosphate modifications, sugar modifications, and 2′ modifications. Recent modifications include replacing the sugar with an alternative structure which has primary and a secondary alcohol groups similar to those of ribose. As used herein, these modified compounds are included within the definition of the term “phosphorus-linked oligomer”.

As used herein, the term “nucleoside phosphoramidite” is intended to denote a mono-, di- or polynuclotide species that has a phosphoramidite functionality attached, preferably at the 3′-terminal position, and which bears a 5′-hydroxyl protecting group.

Such phosphoramidite groups are known in the art to undergo a coupling reaction with the deprotected 5′-hydroxyl of a growing oligomeric chain according to standard synthetic methodologies. See for example Caruthers U.S. Pat. Nos. 4,415,732; 4,458,066; 4,500,707; 4,668,777; 4,973,679; and 5,132,418; and Koster U.S. Pat. Nos. 4,725,677 and Re. 34,069; and Oligonucleotides and Analogues A Practical Approach, Eckstein, F. Ed., IRL Press, New York, 1991, each of the disclosures of which are hereby incorporated by reference in their entirety. Thus, phosphoramidite groups include groups of formula —P(—O—Y)—N(I—pr)₂ where Y is a phosphoryl protecting group useful in phosphoramidite synthesis, such β-cyanoethyl and allyl (See Manoharan, M. et al., Org. Lett. (2000), 2(3), 243-246). Other such phosphoryl protecting groups include methyl, (N-methyl-N-benzoylamino)ethyl, (N-ethyl-N-benzoylamino)ethyl, 2-[N-methyl-N-(4-methoxybenzoyl)-amino]ethyl, 2-(N-isopropyl-N-benzoylamino)ethyl, 2-[N-ethyl-N-(4-methoxybenzoyl)-amino]ethyl, 2-[N-isopropyl-N-(4-methoxybenzoyl)amino]ethyl, 2-[N-methyl-N-(4-dimethylaminobenzoyl)amino]ethyl, 2-[N-ethyl-N-(4-dimethylaminobenzoyl)amino]ethyl, 2-[N-isopropyl-N-(4-dimethylaminobenzoyl)amino]ethyl, 2-(thionobenzoylamino)ethyl, 3-(thionobenzoylamino)propyl, 2-(N-phenylthiocarbamoylamino)ethyl, 2-[(1-naphthyl)carbamoyloxy]ethyl, diphenylsilylethyl, δ-cyanobutenyl, cyano p-xylyl, methyl-N-trifiluoroacetyl ethyl, and acetoxy phenoxy ethyl.

Typically, at the beginning of a solid phase phosphoramidite synthetic regime, a 5′-O-protected nucleoside synthon is first attached to a solid support through a linker. Solid supports are substrates which are capable of serving as the solid phase in solid phase synthetic methodologies, such as those described in Caruthers U.S. Pat. Nos. 4,415,732; 4,458,066; 4,500,707; 4,668,777; 4,973,679; and 5,132,418; and Koster U.S. Pat. Nos. 4,725,677 and Re. 34,069. Linkers are known in the art as short molecules which serve to connect a solid support to functional groups (e.g., hydroxyl groups) of initial synthon molecules in solid phase synthetic techniques. One such linker is a succinamide linker. Other suitable linkers are disclosed in, for example, Oligonucleotides And Analogues A Practical Approach, Ekstein, F. Ed., IRL Press, N.Y, 1991, Chapter 1, pages 1-23.

Solid supports according to the invention include those generally known in the art to be suitable for use in solid phase methodologies, including, for example, controlled pore glass (CPG), oxalyl-controlled pore glass (see, e.g., Alul, et al., Nucleic Acids Research 1991, 19, 1527), TentaGel Support (an aminopolyethyleneglycol derivatized support (see, e.g., Wright, et al, Tetrahedron Letters 1993, 34, 3373)) and Poros (a copolymer of polystyrene/divinylbenzene). Those of skill in the art will appreciate that other solid support methodologies are equally amenable to the methods of the invention.

In accordance with some preferred embodiments of the methods of the invention, after the initial nucleoside synthon is attached to the solid support, and after each subsequent coupling cycle, the 5′-hydroxyl of the previously coupled monomeric or higher order synthon is deprotected with a deprotecting reagent, which can be any of a variety of reagents that are typically used for deprotection of 5′-hydroxyl groups in solid phase oligonucleotide synthesis. Typically, such deprotection reagents includes a protic acid in a solvent, which is typically a halogenated solvent such as dichloromethane or dichloroethane, and, optionally, an additive. Examples of such protic acids include formic acid, acetic acid, chloroacetic acid, dichloroacetic acid, trichloroacetic acid, trifluoroacetic acid, benzenesulfonic acid, toluenesulfonic acid, or phenylphosphoric acid.

In accordance with some preferred embodiments of the invention, the deprotected 5′-hydroxyl of the support bound nucleoside is then typically reacted with (i.e., coupled to) a 5′-protected activated phosphorus compound to produce a covalent linkage therebetween. Where the activated phosphorus compound is a phosphoramidite, a phosphite linkage is produced. Alternatively, other regimes known in the art can be employed to make other internucleoside linkages, for example H-phosphonate linkages and phosphoramidate linkages.

Typically, the 5′-protected phosphordiamidite to be coupled is activated to nucleophilic attack the 5′ hydroxyl of the support bound oligomer by use of an activating agent. It is believed that the activating agent displaces one of the amino groups from the phosphordiamidite, thereby rendering the phosphorus of the phosphordiamidite more susceptible to nucleophilic attack by the 5′ hydroxyl group of the growing nucleotide chain. Any activating agent that can activate the phosphorous to nucleophilic attack without interacting with the growing nucleotide chain may be suitable for use with the present invention. One preferred activating agent is tetrazole. Some commonly used commercially available activating agents are thiotetrazole, nitrotetrazole, and N,N-diisopropylaminohydrotetrazolide. Other suitable activating agents are also disclosed in the above incorporated patents as well as in United States patent 4,725,677 and in Berner, S., Muhlegger, K., and Seliger, H., Nucleic Acids Research 1989, 17:853; Dahl, B. H., Nielsen, J. and Dahl, O., Nucleic Acids Research 1987, 15:1729; and Nielson, J. Marugg, J. E., Van Boom, J. H., Honnens, J., Taagaard, M. and Dahl, O., J. Chem. Research 1986, 26, all of which are herein incorporated by reference.

In some applications of the phosphoramidite technique, each coupling step is followed by a sulfurization step or oxidation step to produce a phosphorothioate or phosphodiester linkage. Useful oxidizing agents according to the present invention include iodine, t-butyl hydroperoxide, or other oxidizing reagents known in the art.

Sulfurizing agents used during oxidation to form phosphorothioate linkages include Beaucage reagent (see e.g. Iyer, R. P., et al., J. Chem. Soc., 1990, 112, 1253-1254, and Iyer, R. P., et al., J. Org. Chem., 1990, 55, 4693-4699); tetraethylthiuram disulfide (see e.g., Vu, H., Hirschbein, B. L., Tetrahedron Lett., 1991, 32, 3005-3008); dibenzoyl tetrasulfide (see e.g., Rao, M. V., et al., Tetrahedron Lett., 1992, 33, 4839-4842); di(phenylacetyl)disulfide (see e.g., Kamer, P. C. J., Tetrahedron Lett., 1989, 30, 6757-6760); 1,2,4-dithiuazoline-5-one (DtsNH) and 3-ethoxy-1,2,4-dithiuazoline-5-one (EDITH) and (see Xu et al., Nucleic Acids Research, 1996, 24, 3643-3644 and Xu et al., Nucleic Acids Research, 1996, 24, 1602-1607); thiophosphorus compounds such as those disclosed in U.S. Pat. No. 5,292,875 to Stec et al., and U.S. Pat. No. 5,151,510 to Stec et al., disulfides of sulfonic acids, such as those disclosed in Efimov et al., Nucleic Acids Research, 1995, 23, 4029-4033, sulfur, sulfur in combination with ligands like triaryl, trialkyl, triaralkyl, or trialkaryl phosphines.

The deprotection and coupling steps, and, optionally, oxidation or sulfurization steps, are repeated using mono-, di- or polymeric activated synthons until at least a portion of the desired base sequence is achieved. In accordance with some preferred embodiments of the invention, the support bound oligomer will contain at least one phosphoryl internucleoside linkage that is not protected. Such an unprotected linkage or linkages can be formed by any of several methods, including, for example, by deprotection of protected phosphoryl groups on the support bound oligomer.

In accordance with some preferred embodiments of the invention, additional phosphoramidite synthesis according to the methods of the invention is performed on the support bound oligomer having at least one unprotected phosphoryl internucleoside linkage. In some preferred embodiments of the invention, the support bound oligomer is washed with a solution containing a neutralizing agent of the invention. This is followed by coupling of a monomeric or higher order nucleoside phosphoramidite. In other preferred embodiments of the invention, the aforementioned washing step is not performed, but rather the support bound oligomer is contacted with a solution that, in addition to the monomeric or higher order nucleoside phosphoramidite to be coupled, also contains has a neutralizing agent of the invention included therein.

In some preferred embodiments, the neutralizing agent of formula D⁺E⁻ is formed in situ from a 1H-tetrazole and an aliphatic amine, an aliphatic heterocyclic amine, an aromatic amine, an aromatic heterocyclic amine, or a guanidine, preferably in about equimolar amounts. While not wishing to be bound by a particular theory, it is believed that where the neutralizing agent is an amine, residual acidity present on the solid support aids in the formation of the protonated form of the amine.

In some preferred embodiments of the invention, concentration of neutralizing agent is from about 0.05M to about 0.15M, with from about 0.08M to about 0.12M being more preferred, with from about 0.09M to about 0.1 M being more preferred, and with about 0.10M being especially preferred.

At the end of desired synthesis, the completed oligomer is cleaved from the solid support. Cleavage is achieved by any of the standard methods in the art, such as, for example, with concentrated ammonium hydroxide. In some preferred embodiments, the conditions for cleavage from the solid support also removes protecting groups from internucleoside linkages, and from the constituent nucleobases.

The methods of the present invention can be used for the synthesis of phosphorus-linked oligonucleotides having both naturally occurring and non-naturally occurring constituent groups. For example, the present invention can be used to synthesize phosphorus-linked oligomers having phosphodiester, phosphorothioate, phosphorodithioate, or H-phosphonate linkages, or having mixtures of such linkages, and which have naturally occurring pentose sugar components such as ribose and deoxyribose, and their substituted derivatives, as well as other sugars known to substitute therefor in oligonucleotide analogs.

The constituent sugars and nucleosidic bases (“nucleobases”) of the phosphorus-linked oligomers can be naturally occurring or non-naturally occurring. Non-naturally occurring sugars and nucleosidic bases are typically structurally distinguishable from, yet functionally interchangeable with, naturally occurring sugars (e.g. ribose and deoxyribose) and nucleosidic bases (e.g., adenine, guanine, cytosine, thymine). Thus, non-naturally occurring nucleobases and sugars include all such structures which mimic the structure and/or function of naturally occurring species, and which aid in the binding of the oligonucleotide to a target, or which otherwise advantageously contribute to the properties of the oligonucleotide.

The methods of the invention are amenable to the synthesis of phosphorus-linked oligomers having a variety of substituents attached to their 2′-positions. As used herein the term “sugar substituent group” or “2′-substituent group” includes groups attached to the 2′ position of the ribosyl moiety with or without an intervening oxygen atom. 2′-Sugar modifications amenable to the present invention include fluoro, chloro, bromo, O-alkyl, O-alkylamino, O-alkylalkoxy, protected O-alkylamino, O-alkylamino-alkyl, O-alkyl imidazole, and polyethers of the formula (O-alkyl)m, where m is 1 to about 10. Preferred among these polyethers are linear and cyclic polyethylene glycols (PEGs), and PEG-containing groups, such as crown ethers, and other reported substituent groups. See, Ouchi et al., Drug Design and Discovery 1992, 9, 93; Ravasio et al., J. Org. Chem. 1991, 56, 4329; and Delgardo et al., Critical Reviews in Therapeutic Drug Carrier Systems 1992, 9, 249. Each of the foregoing references is hereby incorporated by reference in its entirety. Further sugar substituent groups are disclosed by Cook (Anti-Cancer Drug Design, 1991, 6, 585-607). Fluoro, O-alkyl, O-alkylamino, O-alkyl imidazole, O-alkylaminoalkyl, and alkyl amino substituents are described in U.S. patent application Ser. No. 08/398,901, filed Mar. 6, 1995, entitled “Oligomeric Compounds having Pyrimidine Nucleotide(s) with 2′ and 5′ Substitutions,” hereby incorporated by reference in its entirety.

Additional 2′ sugar modifications amenable to the present invention include 2′-SR and 2′-NR2 groups, where each R is, independently, hydrogen, a protecting group or substituted or unsubstituted alkyl, alkenyl, or alkynyl. 2′-SR nucleosides are disclosed in U.S. Pat. No. 5,670,633, issued Sep. 23, 1997, hereby incorporated by reference in its entirety. The incorporation of 2′-SR monomer synthons are disclosed by Hamm et al., J. Org. Chem., 1997, 62, 3415-3420. 2′-NR2 nucleosides are disclosed by Goettingen, M., J. Org. Chem., 1996, 61, 6273-6281; and Polushin et al., Tetrahedron Lett., 1996, 37, 3227-3230. Further representative 2′-O-sugar modifications amenable to the present invention include those having one of formula I or II:

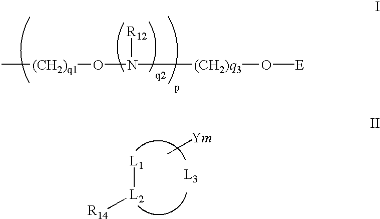

wherein:

E is C₁-C₁₀ alkyl, N(R₁₂)(R₁₃) or N═C(R₁₂)(R₁₃);

each R₁₂ and R₁₃ is, independently, H, C₁-C₁₀ alkyl, a nitrogen protecting group, or R₁₂ and R₁₃, together, are a nitrogen protecting group or are joined in a ring structure that includes at least one additional heteroatom selected from N and O;

R₁₄ is OX₁, SX₁, or N(X₁)₂;

each X₁ is, independently, H, C₁-C₈ alkyl, C₁-C₈ haloalkyl, C(═NH)N(H)Z₁, C(═O)N(H)Z₁ or OC(═O)N(H)Z₁;

Z₁ is H or C₁-C₈ alkyl;

L₁, L₂ and L₃ comprise a ring system having from about 4 to about 7 carbon atoms or having from about 3 to about 6 carbon atoms and 1 or 2 heteroatoms, said heteroatoms being selected from oxygen, nitrogen and sulfur, wherein said ring system is aliphatic, unsaturated aliphatic, aromatic, or saturated or unsaturated heterocyclic;

Yₘ is C₁-C₁₀ alkyl or haloalkyl, C₂-C₁₀ alkenyl, C₂-C₁₀ alkynyl, C₆-C₁₄ aryl, N(R₁₂)(R₁₃) OR₁₂, halo, SR₁₂ or CN;

each q₁ is, independently, an integer from 2 to 10;

each q₂ is 0 or 1;

p is an integer from 1 to 10; and

q₃ is an integer from 1 to 10;

provided that when p is 0, q3 is greater than 1.

In accordance with some preferred embodiments of the methods of the invention, oligomers can be prepared having regions which differ with respect to substituent bound to the 2′-position. Thus, in certain preferred embodiments, the methods of the invention are used to prepare so called “gapped” oligomers wherein, for example, the central portion of the oligomer contains a different 2′-substituent that the regions that form the 5′ and 3′ ends of the oligomer. Thus, it will be appreciated that the methods of the invention find applicability in the preparation of oligomers that have a plurality of such regions differing in 2′-substituent.

The methods of the invention also can be used to prepare oligomers having monomeric subunits that contain O-substitutions of the sugar (for example, ribose or deoxyribose) rings. Representative O-substitutions on the ribosyl ring include S, CH₂, CHF, and CF₂, see, e.g., Secrist, et al., Abstract 21, Program & Abstracts, Tenth International Roundtable, Nucleosides, Nucleotides and their Biological Applications, Park City, Utah, Sep. 16-20, 1992.

Representative nucleobases suitable for use in the methods of the invention include adenine, guanine, cytosine, uridine, and thymine, as well as other non-naturally occurring and natural nucleobases such as xanthine, hypoxanthine, 2-aminoadenine, 6-methyl and other alkyl derivatives of adenine and guanine, 2-propyl and other alkyl derivatives of adenine and guanine, 5-halo uracil and cytosine, 6-azo uracil, cytosine and thymine, 5-uracil (pseudo uracil), 4-thiouracil, 8-halo, oxa, amino, thiol, thioalkyl, hydroxyl and other 8-substituted adenines and guanines, 5-trifluoromethyl and other 5-substituted uracils and cytosines, 7-methylguanine. Further naturally and non naturally occurring nucleobases include those disclosed in U.S. Pat. No. 3,687,808 (Merigan, et al.), in chapter 15 by Sanghvi, in Antisense Research and Application, Ed. S. T. Crooke and B. Lebleu, CRC Press, 1993, in Englisch et al., Angewandte Chemie, International Edition, 1991, 30, 613-722 (see especially pages 622 and 623, and in the Concise Encyclopedia of Polymer Science and Engineering, J. I. Kroschwitz Ed., John Wiley & Sons, 1990, pages 858-859, Cook, P. D., Anti-Cancer Drug Design, 1991, 6, 585-607. The terms “nucleosidic base” and “nucleobase” are further intended to include heterocyclic compounds that can serve as nucleosidic bases, including certain ‘universal bases’ that are not nucleosidic bases in the most classical sense, but function similarly to nucleosidic bases. One representative example of such a universal base is 3-nitropyrrole.

The methods of the present invention use labile protecting groups to protect various functional moieties during synthesis. Protecting groups are used ubiquitously in standard oligonucleotide synthetic regimes for protection of several different types of functionality. In general, protecting groups render chemical functionality inert to specific reaction conditions and can be appended to and removed from such functionality in a molecule without substantially damaging the remainder of the molecule. See, e.g., Green and Wuts, Protective Groups in Organic Synthesis, 2d edition, John Wiley & Sons, New York, 1991. Representative protecting groups useful to protect nucleotides during synthesis include base labile protecting groups and acid labile protecting groups. Base labile protecting groups are used to protect the exocyclic amino groups of the heterocyclic nucleobases. This type of protection is generally achieved by acylation. Two commonly used acylating groups for this purpose are benzoylchloride and isobutyrylchloride. These protecting groups are stable to the reaction conditions used during oligonucleotide synthesis and are cleaved at approximately equal rates during the base treatment at the end of synthesis.

Hydroxyl protecting groups typically used in oligonucleotide synthesis may be represented by the following structure:

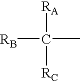

wherein each of R_{A}, R_{B} and R_{C} is an unsubstituted or mono-substituted aryl or heteroaryl group selected from phenyl, naphthyl, anthracyl, and five or six membered heterocylic rings with a single heteroatom selected from N, O and S, or two N heteroatoms, including quinolyl, furyl, and thienyl; where the substituent is selected from halo (i.e., F, Cl, Br, and I), nitro, C₁-C₄-alkyl or alkoxy, and aryl, aralkyl and cycloalkyl containing up to 10 carbon atoms; and wherein R₂ and R₃ may each also be C₁-C₄-alkyl or aralkyl or cycloalkyl containing up to 10 carbon atoms.

In preferred embodiments of the invention, the 5′-protecting group is trityl, monomethoxy trityl, dimethoxytrityl, trimethoxytrityl, 2-chlorotrityl, DATE, TBTr, Pixyl or Moxyl, with trityl, monomethoxy trityl, dimethoxy trityl, 9-phenylxanthine-9-yl (Pixyl) or 9-(p-methoxyphenyl)xanthine-9-yl MOX) being more preferred, and with dimethoxy trityl being especially preferred.

Phosphorus linked oligomers produced by the methods of the invention will preferably be hybridizable to a specific target oligonucleotide. Preferably, the phosphorus linked oligomers produced by the methods of the invention comprise from about 1 to about 100 monomer subunits. It is more preferred that such compounds comprise from about 10 to about 30 monomer subunits, with 15 to 25 monomer subunits being particularly preferred.

As will be recognized, the process steps of the present invention need not be performed any particular number of times or in any particular sequence. Additional objects, advantages, and novel features of this invention will become apparent to those skilled in the art upon examination of the following examples thereof, which are not intended to be limiting.

### EXAMPLES

Materials and Methods

Anhydrous MeCN (water content <0.001%) was purchased from Burdick and Jackson (Muskegon, Mich.). Standard phosphoramidites 2 and 24-26, thymidine H-phosphonate 27, and ancillary reagents for oligonucleotide synthesis were purchased from Glen Research (Virginia). 2′-O-(2-Methoxyethyl) ribonucleoside phosphoramidites 20-23 were obtained from Proligo Biochemie GmbH (Hamburg, Germany). All other reagents and dry solvents were purchased from Aldrich and used without further purification. Tentagel and controlled pore glass versions of the solid support 1 were synthesized as previously reported (See Pon, R. T. and Yu, S. Nucleic Acids Res. 1997, 25, 3629-3635).

³¹P NMR spectra in gel phase were recorded at 80.950 MHZ. The spectra were recorded using 10 to 15 mg of solid supports 3-8 loaded at 208 μmol g⁻¹ and CD₃CN as a liquid phase with the spinning switched off. With the phosphate-unprotected oligonucleotides 4-8, the best resolution was obtained when an amine was added to the liquid phase (1M piperidine for 4 or 5% Py for 5-8).

HPLC Techniques.

Crude 9a, 11a, 16a, 18a, and 32a and 32b were analyzed and isolated on a DeltaPak C18 column (15 μm; 300 Å; 3.8×300 mm) eluting with a linear gradient from 0 to 60% B in 40 min at a flow rate of 1.5 mL min⁻¹. Oligonucleotides 9b, 11b, 16b, and 18b were analyzed using a linear gradient from 0 to 60% B in 30 min. 0.1M aq NH₄OAc and 80% aq MeCN were used as buffer A and buffer B, respectively.

Oligonucleotide Synthesis.

The oligonucleotide synthesis was performed on an ABI 380B DNA Synthesizer. The phosphoramidite synthesis was carried out either according to the manufacturer's recommendations (Standard Cycle) or by a modified procedure. Phosphoramidites 2 and 20-26 were used as 0.1M solutions in dry MeCN. For the attachment of phosphoramidites 20-23, the coupling time was extended to 10 min. For preparation of the PS oligonucleotides, 3H-1,2-benzodithiol-3-one 1,1-dioxide (0.05M in MeCN) was used as a sulfur-transfer reagent (See Iyer, R. P.; Phillips, L. R.; Egan, W.; Regan, J. B.; Beaucage, S. L. J. Org. Chem. 1990, 55, 4693-4699). For preparation of 16a and 16b and 18a and 18b by the modified cycle, the neutralizers, LiClO₄ or a tertiary amine (0.1M in MeCN) or mixtures containing 0.1M tertiary amine and 0.1M 1H-tetrazole, were prepared. These were placed in positions 15 or 17 of the synthesizer (PS and PO cycles, respectively). The standard, 1 μmol protocol was modified in two respects. First, the delivery time for the capping reagents and the following wait time were extended to 45 sec each. This reduced dramatically the presence of the 5′-DMT-protected 19-mer and shorter oligonucleotides and thus simplified the calculation of yields for the less efficient syntheses. Secondly, the standard detritylation subroutine was followed by a brief washing with MeCN and flushing with argon. To convert the support-bound oligonucleotide to the required salt, the solution of a neutralizer was next delivered to the columns for 45 sec. Finally, the columns were washed with MeCN and flushed with argon prior to the coupling step, as in the standard protocol.

The H-phosphonate synthesis was carried out according to the manufacturers recommendations. Thymidine 3′-H-phosphonate 27 and the activator, pivaloyl chloride, were used as 0.05M solutions in MeCN-Py (50:50) and 0.2M solutions in MeCN-Py (95:5), respectively.

Solid support-bound 8, 10, 12, and 17a and 17b were deprotected with concentrated aqueous ammonium hydroxide for 30 min at room temperature. Compounds 31a and 31b were deprotected for 6 h at 55° C. The products, 5′-DMT protected crude oligonucleotides 9a and 9b, 11a and 11b, 16a and 16b, 18a and 18b, and 32a and 32b, were analyzed by reverse phase HPLC and characterized by electron-spray LCMS. The modified oligonucleotides 32a and 32b were isolated and desalted by reverse phase HPLC.

### Example 1

Coupling of Phosphoramidite 2 to Unprotected Hexathymidylates Studied by ³¹P NMR on Solid Support.

While not wishing to be bound by any particular theory, it has been previously postulated that an activated phosphoramidite might react with an internucleosidic phosphate diester moiety to form a mixed anhydride, which could be cleaved in the presence of excess 1H-tetrazole to regenerate deoxyribonucleoside phosphorotetrazolide intermediates (See Caruthers, M. H. Kierzek, R., and Tang, J. Y., supra). Indirectly, this has been confirmed by the fact that the dimethoxytrityl responses measured after each elongation cycle were consistent with addition of only one equivalent of phosphoramidite. Because no similar information was available for phosphorothioate oligonucleotides, the reactivity of the solid support-bound oligonucleotides 4a and 5a with the phosphoramidite 2 was first studied.

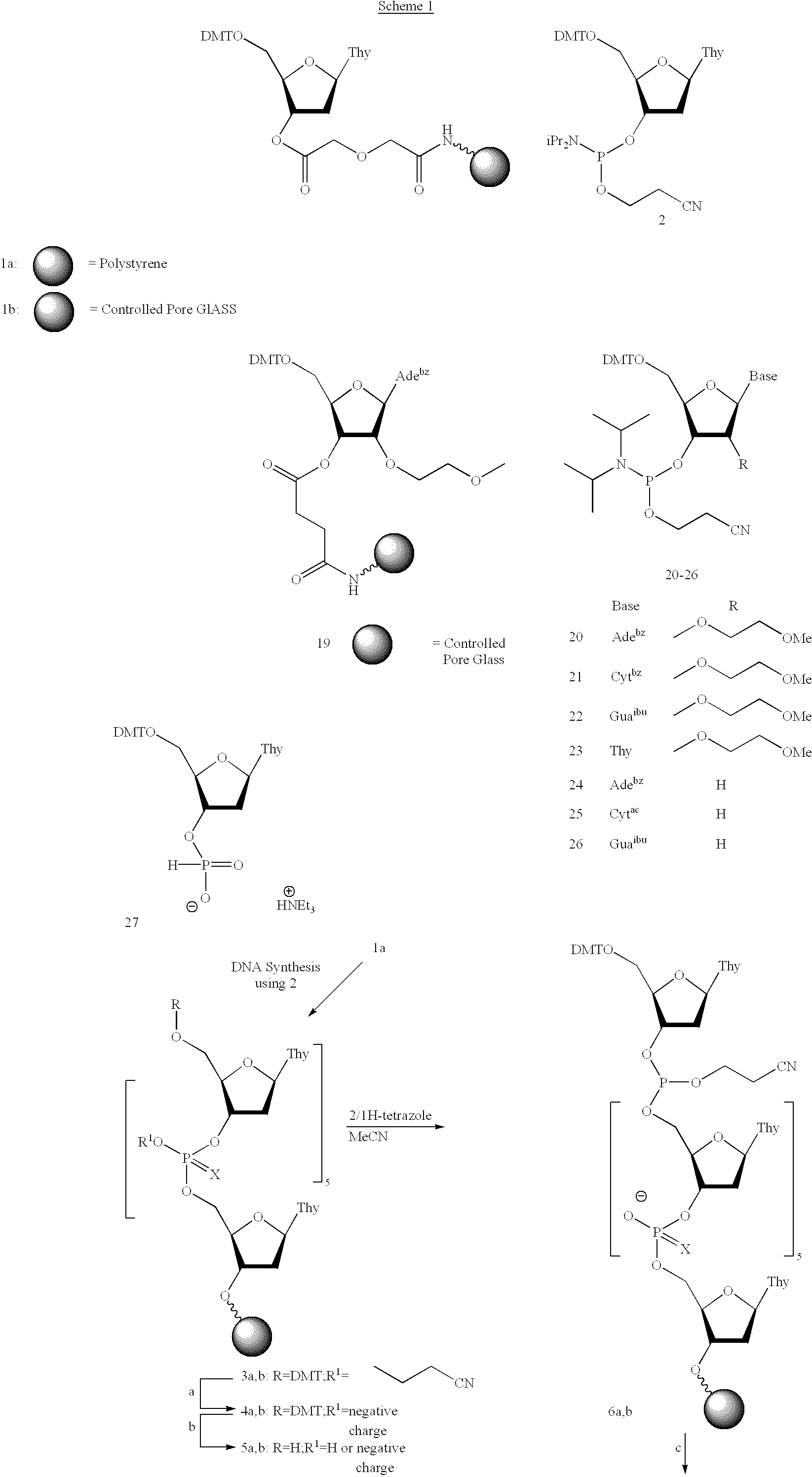

In order to obtain 4a, its 2-cyanoethyl protected precursor 3a was synthesized on a 40 μmol scale on a high-loaded polystyrene support 1a (See Pon et al., supra) using phosphoramidite chemistry (Scheme 1).

The solid support-bound 3a was treated with 1M piperidine in anhydrous MeCN. The progress of the deprotection was monitored by ³¹P NMR in gel phase. After 1.5 h at room temperature, the peak at 67.8 ppm was replaced by a peak at 57.3 ppm, which reflected the conversion of the thionophosphate triester 3a to the corresponding diester 4a in more than 98% yield. At the same time, the diglycolyl linker (See Pon et al., supra) that anchored the oligonucleotide to the solid support was not cleaved to any appreciable extent. On completion of deprotection, 4a was washed with excess MeCN and detritylated to give 5a.

The solid supports 4a and 5a were treated with 2 (0.1M in MeCN) in the presence of 1H-tetrazole for 10 min on a DNA synthesizer followed by excessive washing with MeCN. The ³¹P NMR spectrum of the product obtained from 4a revealed only the peak of the starting material. For 5a, two peaks at 140.6 and 57.5 ppm in a ratio of 1:5.06 were observed, which agreed with the formation of 6a in ca. 99% yield. When sulfurized with 3H-1,2-benzodithiol-3-one 1,1 dioxide (See Iyer et al., supra), 6a gave 7a (peaks at 67.9 and 57.5 ppm) in quantitative yield.

In a similar manner, 4a and 5a were treated with the standard capping mixture (Ac₂O/N-methylimidazole/pyridine/THF) for 30 min in a NMR tube. No apparent changes in the ³¹P NMR spectra of both compounds were observed. When four additional coupling cycles were carried out with 7a, solid support-bound 8a was obtained. In the ³¹P NMR spectrum of 8a, the peaks of the protected and the deprotected PS moieties were displayed at 67.8 and 56.8 ppm, respectively, in a 50:50 ratio. Additionally, a minor peak of desulfurized phosphates was observed at −1.5 ppm and accounted for ca. 2% of the total integration area.

Analogous observations were made when oligonucleotides 3b-8b were synthesized. These experiments confirmed that no stable products are formed between phosphodiester groups and the nucleoside phosphoramidite 2 or Ac₂O in the presence of 1H-tetrazole and N-methylimidazole, respectively. They also demonstrated that the hypothesis holds true for thionophosphate diesters. The solid support-bound compounds 8a and 8b were treated with concentrated aqueous ammonium hydroxide to give 9a and 9b, respectively. The reverse phase HPLC analysis of the crude products suggested an average coupling efficiency that did not exceed 94-95%. In addition, during the synthesis of 8a and 8b we observed some detritylation during the coupling step.

While not wishing to be bound by any particular theory, possible explanations for the low coupling efficiency may be posited using compounds 13-15 in Scheme 2 as illustrations. After the phosphate-unprotected oligonucleotide 13 is detritylated on a DNA synthesizer by treatment with dichloroacetic acid (pKₐ 1.30 in water; See Gould, E. S. Mechanism and Structure in Organic Chemistry; Henry Holt and Company: New York, 1960, p. 201), the phosphodiester backbone may be at least partially converted to an acid 14 (Scheme 2). After washing with MeCN, 14 is treated with a phosphoramidite building block and 1H-tetrazole. In the standard protocols for DNA synthesis, phosphoramidite building blocks are used, depending on the synthetic scale, in a two to tenfold excess over the 5′-hydroxy groups of the support-bound oligonucleotide. Thus, for the 11-mer oligonucleotide 14, the concentration of the support-bound dinucleoside phosphoric acid in the reaction volume may exceed or at least be comparable to the concentration of the phosphoramidite. This may lead to the unwanted partial detritylation and inactivation of the monomer and result in lower coupling yields. Thus, in accordance with the present invention, prior to the coupling step, 14 was neutralized, i.e., converted to a salt 15 using a base or a salt of the base. Preferably, the counterion, BH⁺ or M⁺, should be inert towards nucleoside phosphoramidites.

### Example 2

Coupling of Phosphoramidite 2 to Oligonucleotides with Neutralized Phosphodiester Groups.

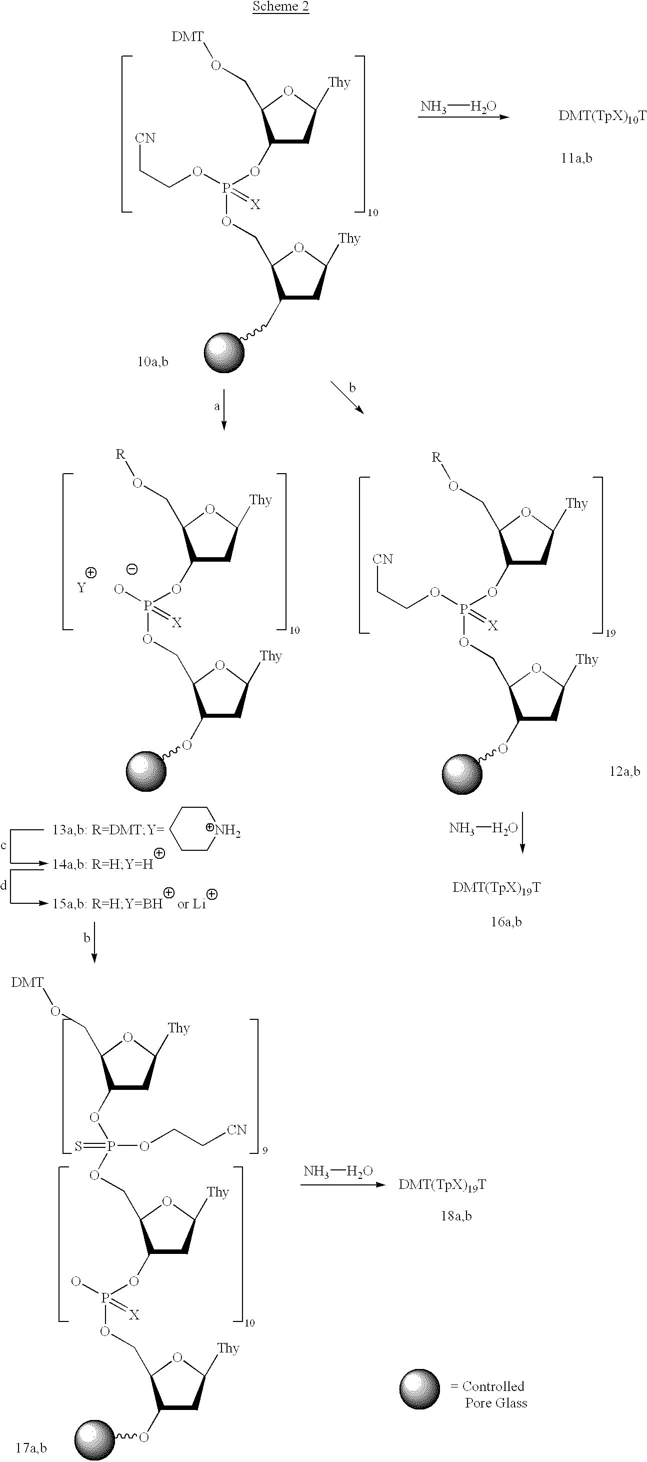

In order to study the effect of the counterion on the efficiency of the phosphoramidite coupling, model experiments were carried out as presented in Scheme 2.

Starting from 1b, protected 3′-segments 10a and 10b were first synthesized on a 30 μmol scale in a standard manner. Small aliquots of 10a and 10b (0.5 μmol) were conventionally deprotected to give 11a and 11b, which were characterized by ESMS and HPLC. Approximately one half of 10a and 10b was converted to the unprotected 3′-segments 13a and 13b as described for compound 8a. With these as starting material, further chain elongation was carried out on a 1 μmol scale until protected 9-mer 5′-segments were assembled to give 17a and 17b and 18a and 18b. For comparison, control samples of the same sequence, 12a and 12b and 16a and 16b, were synthesized from 10a and 10b on a parallel column using the identical scale, protocols and conditions.

In comparison with the experimental design for compound 8, this offered two distinct advantages. First, a longer 11-mer 3′-segment allowed one to observe a more pronounced negative effect of the unprotected phosphate backbone. Secondly, at a high coupling efficiency, assembling a protected 9-mer 5′-segment resulted in greater differences in yields between the experimental and the control samples, 16 and 18, respectively. This led to a more accurate determination of the stepwise yields for the 5′-segment of 16 and 18 and more reliable results regarding the coupling efficiency.

The standard protocol for the DNA synthesis on a 1 μmol scale was modified in two aspects. First, delivery and wait times on the capping step were each extended to 45 sec. This reduced dramatically the abundance of DMT-positive (n−1)-mer and shorter oligonucleotides and thus simplified the calculation of yields for the full-length oligonucleotides. Secondly, the standard detritylation subroutine was followed by a modified washing protocol. To convert the detritylated, support-bound oligonucleotides to the required salt, a solution of a neutralizer, i.e., an organic base or a salt was delivered to the columns for 45 sec. Then, in accordance with the standard protocol, the columns were washed with MeCN, and the coupling subroutine was carried out.

For neutralization of the phosphorothioate and phosphodiester (PS and PO, respctively) backbones, a number of amines, i.e., pyridine (Py), 2,6-lutidine (Lut), 2,4,6-collidine (Col), N-methylnorpholine (NMM), N,N-diisopropylethylamine (DIPEA), and triethylamine (TEA) were used as 0.1M solutions in MeCN. The pK_{BH}+ (MeCN) values of the conjugated acids of these amines covered a wide range from 12 to 18.5, so that a possible dependence of the acidity of a protonated amine on the coupling efficiency could be revealed (for pK_{BH}+ values in MeCN, see: (a) Py, 12,33; Lut, 13.92; Col, 14.77; DMAP, 17.74; DBU, 24.13 (Kaljurand, I.; Bodima, T.; Leito, I.; Koppel, I. A.; Schwesinger, R. J. Org. Chem. 2000, 65, 6202-6208); (b) NMM 15.59 (Izutsu, K. Acid-Base Dissociation Constants in Dipolar Aprotic Solvents; Blackwell Scientific Publ.: Oxford, 1990; 166 pp.); (c) DIPEA 18.00 (estimated; the experimental value for DIPEA does not appear to have been reported); (d) TEA, 18.46 (Coetzee, J. F.; Padmanabhan, G. R. J. Am. Chem. Soc. 1965, 87, 5005-5010); (e) TMG, 23.3 (Schwesinger, R. Nachr. Chem., Tech. Lab. 1990,38, 1214-26)).

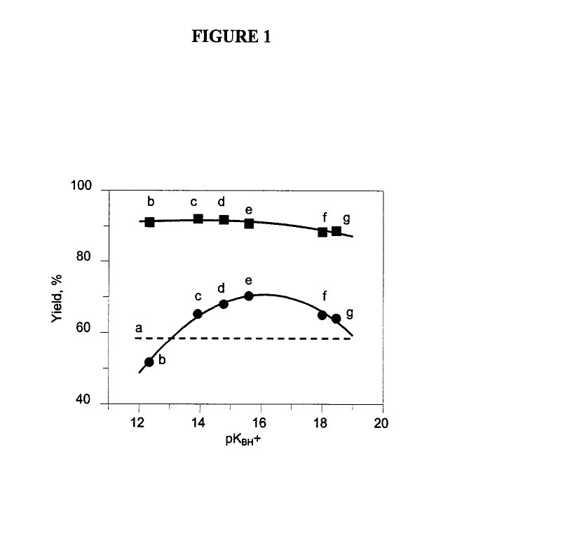

On completion of the synthesis, 12a and 12b and 17a and 17b were treated with concentrated ammonium hydroxide for 30 min to give crude 16a and 16b and 18a and 18b, respectively. These were analyzed by reverse phase HPLC. Average stepwise coupling yields and total yields for the 5′-segments of the full-length oligonucleotides 16a and 16b and 18a and 18b were next calculated. The data obtained are presented in FIG. 1 (16a and 18a) and 3 (16b and 18b). The total yields of the 5′-segments of the oligonucleotides are plotted against the pK_{BH}+ (MeCN) values of the protonated bases that served as counterions for the PS or PO residues. Horizontal grids in FIGS. 1-3 represent the yields 18a and 18b obtained using the standard cycle.

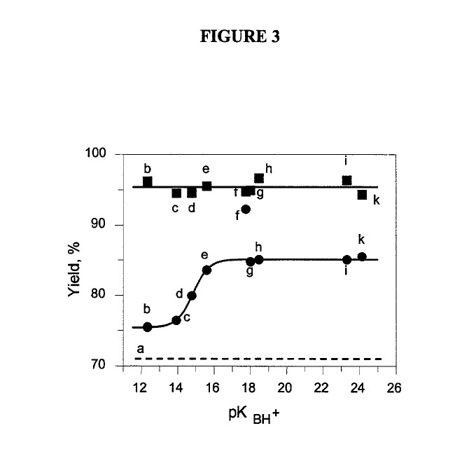

As seen from FIGS. 1 and 3, the yields of 16a and 16b were not influenced by the nature of the neutralizer except when DIPEA and TEA free bases were used for the preparation of 16a (FIG. 1). In agreement with the preliminary results for 9a and 9b, the preparation of 18a and 18b using the standard cycle resulted in significantly lower yields of the products. The stepwise yield of 18b (96.4%) correlated perfectly with the reported value of 96% (See Caruthers, M. H., Kierzek, and Tang, J. T., supra). In contrast, when tertiary amines were used as neutralizers, improved yields of 18a and 18b were obtained. This effect depended on the pK_{BH}+ value of the counterion. As seen in FIG. 1, for the free amines, the yields of 18a reached their maximum around pK_{BH}+ value of protonated NMM. With more basic amines, DIPEA and TEA, lower yields of both 16a and 18a were obtained. While not wishing to be bound by any particular theory, it is possible that amines as strong as DIPEA and TEA might cause a partial decyanoethylation in the course of the neutralization step, which generated additional unprotected phosphates and thus decreased the efficiency of the synthesis for both 16a and 18a.

To confirm that the use of a free base was not mandatory for an efficient cation exchange between the solid support-bound PS or P0 moieties and the solution of a neutralizing agent, solutions of DBU (See Kaljurand et al., supra) in MeCN were mixed with different concentrations of 1H-tetrazole (Tet) or with AcOH. As an example of inorganic salts, LiClO₄, which is readily soluble in MeCN, was tested. These agents were used as neutralizers in the synthesis of 16a and 18a as described above for the free amines.

The highest yields of 16a and 18a were obtained with a mixture of 0.1M DBU and 0.1M Tet (93.3 and 75.4%, respectively). Very similar results were obtained for both oligonucleotides when a slight excess (0.11M) of Tet was present . In contrast, the yields of 16a and 18a were dramatically lower (by 29 and 11%, respectively) when excess DBU was used (0.10 M DBU+0.09 M Tet). Similarly, using an equimolecular mixture of DBU and glacial AcOH or a solution of LiClO₄ reduced the yield of 16a by more than 20%.

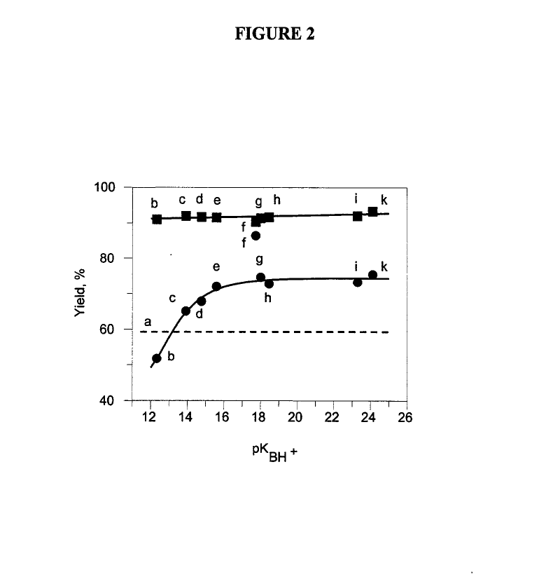

A number of strong amines, i.e., NMM, 4-dimethylaminopyridine (DMAP) (See Kaljurand et al., supra), DIPEA, TEA, and N,N,N′,N′-tetramethylguanidine (TMG) (See schwesinger et al., supra) were tested as equimolecular mixtures with Tet. At 0.1M concentration, all salts with Tet were readily soluble in dry MeCN and thus could be used safely on a DNA synthesizer. As seen in FIGS. 2 and 3, the use of these agents had no adverse effect on the synthesis of 16a and 16b. Moreover, the yields of 18a and 18b obtained with 1H-tetrazolides of NMM, DIPEA, and TEA were higher than with the corresponding free amines. With neutralizing agents other than the mixture of DMAP and Tet, the yields of 18a and 18b reached a plateau around the PK_{BH}+ value of protonated DIPEA and then remained independent of the acidity of the protonated amine.

The pattern of the experimental curves was characteristic for the titration, which is most apparent for 18b (FIG. 3). In addition, the datapoints in FIG. 3 were best fitted using the Henderson-Hasselbach equation (See, for example: Atkins, P. W. Physical Chemistry, 3rd Ed.; W. H. Freeman and Co.: New York, 1985, p. 280) which, in the present case, was transformed to eq (1):

$${pK}_{{BH}^{+}} = ( 14.83 \pm 0.05 ) + lg ( \frac{Y - {Y}_{min}}{{Y}_{max} - Y} ) ; \\ where \frac{Y - {Y}_{min}}{{Y}_{max} - Y} = \frac{[ {A}^{-} ]}{[ HA ]} \qquad ( 1 )$$

While not wishing to be bound by any particular theory, it would be expected that, at low PK_{BH}+ of a neutralizing base, the internucleosidic moiety may present itself as the O,O′-dinucleoside phosphoric acid (HA). At high pK_{BH}+, it is mostly ionized to form the corresponding phosphate anion (A⁻). It can be seen from FIG. 3 that both HA and A⁻ display a negative effect on the yield of 18b (Y, %). However, the effect of A⁻ is less pronounced, which is reflected in a higher yield of 18b (Yₘₐₓ=85.1%) at high pK_{BH}+. In contrast, the species HA that are dominant at low PK_{BH}+ reduce the yield of 18b more substantially (Yₘᵢₙ=75.4%).

It has been reported recently that tertiary ammonium azolide salts provide a more efficient catalysis in alcoholysis of dialkyl tetrazolylphosphonite than the corresponding azoles or tertiary amines (See Nurminen, E. J.; Mattinen, J. K.; Lonnberg, H. J. Chem. Soc. Perkin Trans. 2 1999, 2551-2556). The catalytic effect of the salts correlated with the difference in the pK values of the acid and the base components, with the salts of stronger protolytes being more powerful catalysts. Therefore, while not wishing to be bound by a particular theory, it is also possible that the observed improvement in the yields of 18a and 18b arises, at least partially, from the catalytic effect of tertiary ammonium salts on the phosphoramidite coupling.

The yields of 18a and 18b that were obtained using DMAP-Tet departed from the general trend and were markedly higher than might be expected from the pK_{BH}+ value of the protonated DMAP (FIGS. 2 and 3). The mean total yields of the 5′-segments of 18a and 18b were 86.4±0.2% and 92.2±0.3% (n=4), or only 4 and 3% lower than the yields of 16a and 16b, respectively. These data corresponded to the average stepwise yield of 98.4 and 99.1% for the PS and PO coupling cycles.

While not wishing to be bound by any particular theory, one can explain the effect of the DMAP 1H-tetrazolide by one considering the outstanding catalytic ability of DMAP in nucleophilic reactions. It is believed that an activated phosphoramidite may form a mixed anhydride with an internucleosidic phosphate diester group. In the presence of excess 1H-tetrazole, this intermediate could be cleaved to regenerate the activated phosphoramidite, nucleoside phosphorotetrazolide (See (a) Caruthers, M. H., Kierzek, and Tang, J. T.; and (b) Bruzik et al., supra). As seen from the results of the ³¹P NMR studies, the mixed anhydride is a short-lived intermediate that is not observed directly. However, by forming the mixed anhydride, the internucleosidic phosphates may efficiently compete with the 5′-hydroxy groups for the reactive species. Accordingly, the positive effect of DMAP may consist in catalyzing the reverse reaction, i.e., the regeneration of the nucleoside phosphorotetrazolide. Alternatively, DMAP may catalyze the coupling to the 5′-hydroxy groups in a similar manner to the reported observations concerning the synthesis of oligoribonucleotides (See Pon, R. T. Tetrahedron Lett. 1987, 28, 3643-3646).

The results presented herein show that the efficiency of the phosphoramidite coupling to phosphate unprotected oligonucleotides is lower than with the standard, protected oligonucleotides. However, the efficiency of the synthesis can be improved by compounds of the invention, particularly 0.1M DMAP 1H-tetrazolide in MeCN, for the neutralization of the PS or PO backbone. Under optimized conditions, stepwise yields as high as 98+% were obtained.

### Example 3

Synthesis of Modified Oligonucleotides using Phosphoramidite and H-Phosphonate Methods in Succession.

The practical utility of phosphoramidite coupling to phosphate-unprotected oligonucleotides was demonstrated by preparing chimeric antisense oligonucleotides 32a and 32b against human MDM2 mRNA. As depicted in Scheme 3, compounds 32a and 32b comprised three segments consisting of PS and nucleoside-3′-phosphoramidate (PN) linkages. In addition, six 2′-O-(2-methoxyethyl) ribonucleoside (MOE) residues (see Martin, P. Helv. Chim. Acta 1995, 78, 486-504) were introduced at each terminus of 32a and 32b.

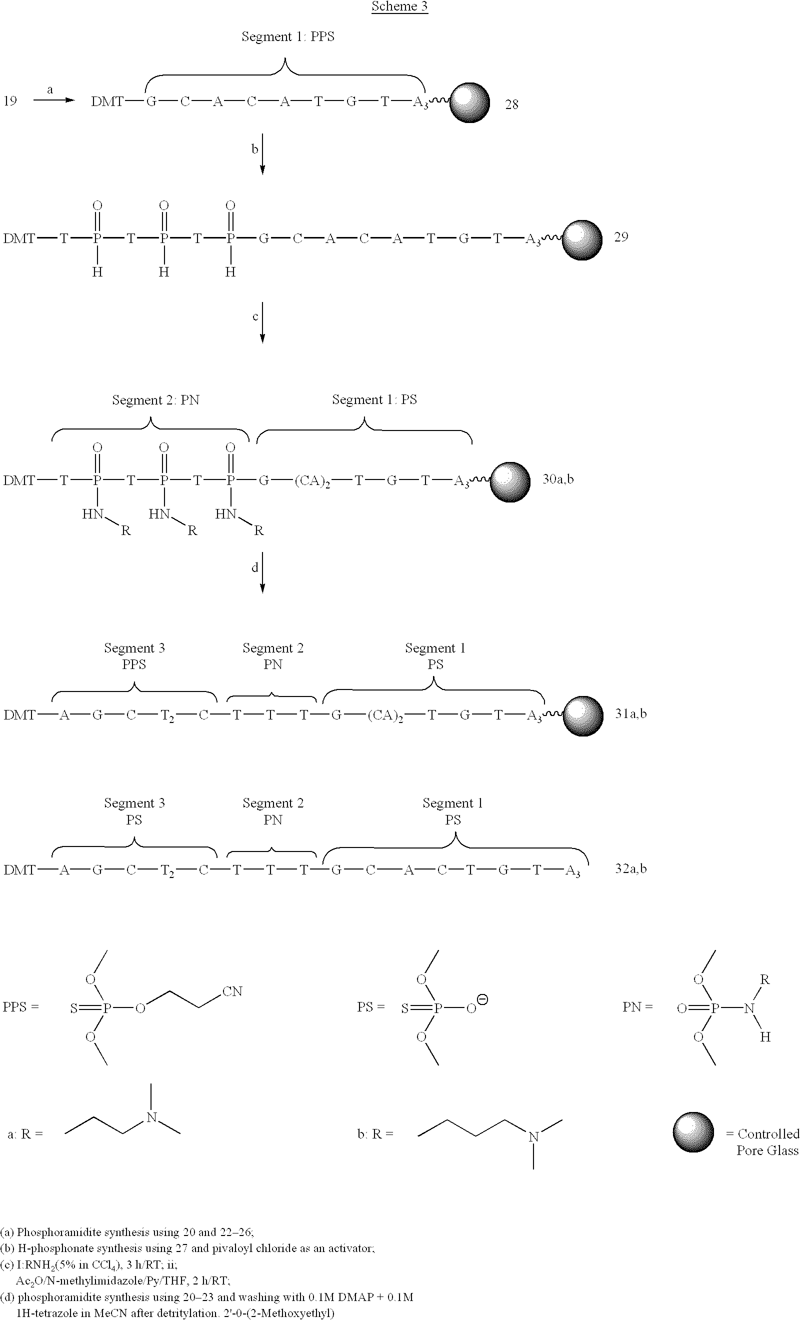

Starting from 19, a solid support-bound, protected oligonucleotide 28, whose thionophosphate triester internucleosidic linkages (PPS) were protected with 2-cyanoethyl groups, was first synthesized by the phosphoramidite method using the commercially available building blocks 20 and 22-26. The synthesis then proceeded with assembly of the segment 2 by the H-phosphonate method using 27 as a building block and pivaloyl chloride as the activator. The product 29 was converted to 30a and 30b by oxidative amidation of H-phosphonate linkages with a solution of a primary amine in CCl₄ as described previously (See Maier, M. A.; Guzaev, A. P.; Manoharan, M. Org. Lett. 1999, 2, 1819-1822). Simultaneously, the 2-cyanoethyl protecting groups were removed to convert the PPS groups in segment 1 to the deprotected thionophosphate diester internucleosidic linkages, PS. In addition, treatment with primary amines partially removed, the base protecting groups. These were restored by acylation with the standard capping reagent (Ac₂O/N-methylimidazole/Py/THF) for 2 h.

It has been previously reported that the chain elongation by the H-phosphonate approach can be carried out efficiently with solid support-bound, phosphate unprotected oligonucleotides (See Gryaznov, S. M.; Potapov, V. K. Tetrahedron Lett. 1991, 32, 3715-3718). Therefore, the synthesis of the oligonucleotide analogs of 32a and 32b could be completed using the H-phosphonate building blocks. However, the H-phosphonate counterparts of the building blocks 20-23 are not available commercially and hence they have to be synthesized, purified, and characterized as described previously. 13 Similarly, many other nucleosidic and non-nucleosidic building blocks for the preparation of modified oligonucleotides are less commonly used and are commercially available only as 2-cyanoethyl phosphoramidites.

Using the methods of the present invention, one can avoid the synthesis of unusual H-phosphonate building blocks. Starting from 30a and 30b, the synthesis was resumed by the phosphoramidite method with 20-23 as building blocks. The synthesis was carried out using the modified elongation cycle where detritylation was followed by washing with the neutralizer (0.1M DMAP and 0.1M Tet) as described above for 18a and 18b. The products 31a and 31b were deprotected with concentrated ammonium hydroxide in a conventional manner to give 32a and 32b. These were isolated by reverse phase HPLC in 33% and 36% yield, respectively, and characterized by ESMS. By comparison, when the standard cycle was used, the yield of 32a was only 12%.

Calculations.

The accurate calculation of the stepwise yields in oligonucleotide synthesis requires the use of a complex mathematical model. See (a) Foldes-Papp, Z.; Baumann, G.; Birch-Hirschfeld, E.; Eickhoff, H.; Greulich, K. O.; Kleinschmidt, A. K.; Seliger, H. Biopolymers 1998, 45, 361-379; (b) Foeldes-Papp, Z.; Birch-Hirschfeld, E.; Eickhoff, H.; Baumann, G.; Peng, W. G.; Biber, Thomas; Seydel, R.; Kleinschmidt, A. K.; Seliger, H. J. Chromatogr., A 1996, 739, 431-447; (c) Foeldes-Papp, Z.; Peng, W. G.; Seliger, H.; Kleinschmidt, A. K. J. Theor. Biol. 1995, 174, 391-408. Herein, a simpler (and less accurate) approach was used. For calculations of stepwise yields for normal and phosphate-deprotected oligonucleotides, it was assumed that the unreacted 5′-hydroxy functions were acetylated quantitatively on the capping step. Since, even with an extended capping protocol, this condition is not completely met, the calculated values are somewhat higher than the actual yields. Apparently, the lower the coupling efficiency is, the more abundant the uncapped DMT-positive shortmers are. Thus, the calculated values are less accurate when the coupling yields are low. In contrast, for highly efficient syntheses, the calculated results are very accurate.

The homobasic oligonucleotides were assembled on a nucleosidic solid support in two segments, n and m phosphate residues in length, in the average stepwise coupling yields x and y for the 3′- and 5′-segments, respectively. On completing the synthesis of the 3′-segment, an aliquot of solid support-bound oligonucleotide was withdrawn, deprotected with aqueous ammonium hydroxide, and analyzed by HPLC. From the HPLC-trace, the ratio of the integrated areas for full-length, DMT-On product and DMT-Off shortmers, R_{(3′-sgm)} was determined. The equation (S1) was solved numerically for x:

$${R}_{( {3}^{′} - sgm )} - \frac{( n + 1 ) {x}^{n}}{( 1 - x ) {\sum }_{n = 1}^{n} {nx}^{n - 1}} = 0 \qquad ( S1 )$$

On completing the synthesis of the 5′-segment, the solid support-bound oligonucleotide was treated as described above. In a similar manner, the ratio of the integrated areas for the full-length, DMT-On product and the DMT-Off shortners, R_{(5′-sgm)} was determined. The equation (S2) where x is known from solving the equation (S1) above was solved numerically for y:

$${R}_{( {5}^{′} - sgm )} - \frac{( m + n + 1 ) {x}^{n} {y}^{m}}{( 1 - x ) {\sum }_{n = 1}^{n} {nx}^{n - 1} + ( 1 - y ) {x}^{n} {\sum }_{m = 1}^{m} ( n + m ) {y}^{m - 1}} = 0 \qquad ( S2 )$$

The total yield of the 5′-segment was found as (y^{m′} 100%). The calculated values for the total yield and the average stepwise yield (y′ 100%) are presented in Table 1.

TABLE S1
Calculated Average Stepwise and Total Yields for the 5'-Segments of
Oligonucleotides 16a and 16b and 18a and 18b.

| | Yield, %^{a} | | | | | | | |
|---|---|---|---|---|---|---|---|---|
| Washing | 16a, per | 16a, | 18a, per | 18a, | 16b, per | 16b, | 18b, per | 18b, |
| Protocol | step | total | step | total | step | total | step | total |
| Standard | 99,17 | 92,79 | 93,78 | 56,08 | 99,43 | 95,00 | 96,33 | 71,42 |
| cycle | | | | | | | | |
| 0.1 M Py | 98,96 | 91,02 | 92,94 | 51,74 | 99,58 | 96,25 | 96,92 | 75,44 |
| 0.1 M Lut | 99,08 | 92,02 | 95,35 | 65,13 | 99,38 | 94,54 | 97,05 | 76,38 |
| 0.1 M Col | 99,05 | 91,76 | 95,79 | 67,90 | 99,38 | 94,59 | 97,54 | 79,93 |
| 0.1 M NMM | 98,92 | 90,69 | 96,15 | 70,23 | — | — | — | — |
| 0.1 M | 98,63 | 88,36 | 95,30 | 64,85 | — | — | — | — |
| DIPEA | | | | | | | | |
| 0.1 M TEA | 98,67 | 88,67 | 95,15 | 63,93 | — | — | — | — |
| 0.1 M NMM; | 99,03 | 91,60 | 96,42 | 72,00 | 99,50 | 95,55 | 98,03 | 83,57 |
| 0.1 M | | | | | | | | |
| 1H-tetrazole | | | | | | | | |
| 0.1 M | 98,883 ± | 90,385 ± | 98,393 ± | 86,436 ± | 99,403 ± | 94,756 ± | 99,107 ± | 92,245 ± |
| DMAP; 0.1 M | 0,023 | 0,186 | 0,023 | 0,179 | 0,011 | 0,297 | 0,013 | 0,339 |
| 1H-tetrazole^{b} | | | | | | | | |
| 0.1 M | 99,01 | 91,44 | 96,80 | 74,61 | 99,42 | 94,94 | 98,18 | 84,76 |
| DIPEA; 0.1 M | | | | | | | | |
| 1H-tetrazole | | | | | | | | |
| 0.1 M TEA; | 99,04 | 91,68 | 96,53 | 72,77 | 99,62 | 96,66 | 98,22 | 85,06 |
| 0.1 M | | | | | | | | |
| 1H-tetrazole | | | | | | | | |
| 0.1 M DBU; | 99,23 | 93,28 | 96,91 | 75,39 | 99,59 | 96,36 | 98,27 | 85,50 |
| 0.1 M | | | | | | | | |
| 1H-tetrazole | | | | | | | | |
| 0.1 M DBU; | 95,23 | 64,41 | 95,58 | 66,59 | — | — | — | — |
| 0.09 M | | | | | | | | |
| 1H-tetrazole | | | | | | | | |
| 0.1 M DBU; | 99,12 | 92,36 | 96,75 | 74,30 | — | — | — | — |
| 0.11 M | | | | | | | | |
| 1H-tetrazole | | | | | | | | |
| 0.1 M DBU; | 95,96 | 68,99 | 96,26 | 70,95 | — | — | — | — |
| 0.1 M AcOH | | | | | | | | |
| 0.1 M TMG; | 99,08 | 92,05 | 96,60 | 73,23 | 99,35 | 94,34 | 98,22 | 85,06 |
| 0.1 M | | | | | | | | |
| 1H-tetrazole | | | | | | | | |
| 0.1 M LiClO₄ | 93,81 | 57,24 | 92,62 | 50,18 | 89,18 | 35,69 | 85,87 | 25,38 |

^{a.}Mean values were calculated from the results of two independent experiments.
^{b.}Mean values and standard errors were calculated from the results of four independent experiments.

TABLE S2
ESMS Data for oligonucleotides 11a and 11b, 16a and 16b, 18a and 18b,
and 32a and 32b.^{a}

| | | Observed | Expected |
|---|---|---|---|
| Compound | Sequence (5′ → 3′) | Mass | Mass |
| 11a | DMT-(TpS)₁₀T | 3746.73 | 3747.20 |
| 16a, Standard cycle | DMT-(TpS)₁₉T | 6629.25 | 6629.54 |
| 16a, using DMAP-Tet | DMT-(TpS)₁₉T | 6629.09 | 6629.54 |
| 18a, using DMAP-Tet | DMT-(TpS)₁₉T | 6629.19 | 6629.54 |
| 11b | DMT-(TpO)₁₀T | 3586.22 | 3586.53 |
| 16b Standard cycle | DMT-(TpO)₁₉T | 6323.88 | 6324.27 |
| 16b, using DMAP-Tet | DMT-(TpO)₁₉T | 6424.10 | 6324.27 |
| 18b, using DMAP-Tet | DMT-(TpO)₁₉T | 6323.91 | 6324.27 |
| 32a^{b} | DMT-agct₂c | 7776.4 | 7774.8 |
| | (TpN¹)₃ GCACA tgta₃ | | |
| 32b^{b} | DMT-agct₂c | 7819.5 | 7816.9 |
| | (TpN²)₃ GCACA tgta₃ | | |

^{a}2′-deoxy and 2′-O-(2-methoxyethyl) nucleosides are given in upper and lower case, respectively; pS, pO, pN¹, and pN² stand for phosphorothioate, phosphate, N—(N,N-dimethylaminoethyl) phosphoramidate, and N—(N,N-dimethylaminopropyl) phosphoramidate internucleosidic linkages.
^{b}Unless it is specified otherwise, the backbone is phosphorothioate

It is intended that each of the patents, applications, printed publications, and other published documents mentioned or referred to in this specification be herein incorporated by reference in their entirety.

Those skilled in the art will appreciate that numerous changes and modifications may be made to the preferred embodiments of the invention and that such changes and modifications may be made without departing from the spirit of the invention. It is therefore intended that the appended claims cover all such equivalent variations as fall within the true spirit and scope of the invention.

What is claimed is:

1. A method comprising reacting a nucleoside phosphoramidite with a support bound oligomer in the presence of a neutralizing agent, said support bound oligomer having at least one unprotected internucleoside linkage selected from the group consisting of phosphate linkages, phosphorothioate linkages, and phosphorodithioate linkages;
wherein said neutralizing agent is:
an aliphatic amine, an aliphatic heterocyclic amine, an aromatic amine, an aromatic heterocyclic amine, a guanidine, or a salt of formula D⁺E⁻ wherein:
D⁺ is a quaternary tetraalkylammonium cation, or a protonated form of an aliphatic amine, an aliphatic heterocyclic amine, an aromatic amine, an aromatic heterocyclic amine, or a guanidine; and
E⁻ is a tetrazolide anion, 4,5-dicyanoimidazolide anion, a substituted orunsubstituted alkylsulfonate anion, a substituted or unsubstituted arylsulfonate anion, tetrafluoroborate anion, hexafluorophosphate anion, or a trihaloacetate anion.

2. The method of claim 1 wherein said neutralizing agent is a salt of formula D⁺E⁻.

3. The method of claim 2 wherein E⁻ is a tetrazolide anion.

4. The method of claim 1 wherein E⁻ is 1H-tetrazolide anion, 5-methylthio-1H-tetrazolide anion, 5-ethylthio-1H-tetrazolide anion or 1-phenyl-5-thiol-1H-tetrazolide anion.

5. The method of claim 1 wherein E⁻ is 1H-tetrazolide anion.

6. The method of claim 3 wherein D⁺ is a protonated form of any of an alkyl, alkenyl or alkynyl amine having from one to about 20 carbons, an aliphatic heterocyclic amine, an aromatic heterocyclic amine, or a guanidine.

7. The method of claim 1 wherein D⁺ is a protonated form of an alkyl amine.

8. The method of claim 3 wherein D⁺ is a protonated form of trimethyl amine, triethyl amine, triisopropyl amine, tributyl amine, triamyl amine, isopropyldimethyl amine, t-butyldimethyl amine, diisopropylethyl amine, or N,N,N′,N′-tetramethyl-1,2-diaminoethane.

9. The method of claim 3 wherein D⁺ is a protonated form of an aliphatic heterocyclic amine.

10. The method of claim 3 wherein D⁺ is a protonated form of any of DBU, N-methylmorpholine, N-methylpyrrolidine, N-methylpiperidine, N,N′-dimethylpiperazine, -ethylpyrrolidine, N-ethylpiperidine, N,N′-diethylpiperazine, 1,5-diazabicyclo[4.3.0]non-5-ene, 1,4-diazabicyclo[2.2.2]octane, or 1,5,7-triazabicyclo[4.4.0]dec-5ene.

11. The method of claim 3 wherein D⁺ is a protonated form of an aromatic heterocyclic amine.

12. The method of claim 3 wherein D⁺ is a protonated form of a mono-, di- or trialkyl pyridine that is optionally substituted with an amino group.

13. The method of claim 3 wherein D⁺ is a protonated form of any of 2,4,6-collidine, 2,6-lutidine, pyridine, 2-methylpyridine, 2,6-diethylpyridine, 2,6-di(t-butyl)pyridine, 4-methyl-2,6-di(t-butyl)pyridine, or 2,4,6-tri(t-butyl)pyridine.

14. The method of claim 3 wherein D⁺ is a protonated form of an alkylamino substituted pyridine.

15. The method of claim 3 wherein D⁺ is a protonated form of 4-dimethylaminopyridine.

16. The method of claim 3 wherein D⁺ is a protonated form of guanidine.

17. The method of claim 3 wherein D⁺ is a protonated form of a tetraalkyl guanidine.

18. The method of claim 3 wherein D⁺ is a protonated form of N,N,N′N′-tetramethylguanidine.

19. The method of claim 3 wherein D⁺ is a quaternary tetraalkylammonium cation.

20. The method of claim 3 wherein D⁺ is a tetramethylammonium, tetraethylammonium, tetrapropylammonium, tetrabutylammonium, trimethyloctylammonium, or triethylbenzylammonium cation.

21. The method of claim 3 wherein E⁻ is 1H-tetrazolide anion.

22. The method of claim 1 wherein E⁻ is 4,5-dicyanoimidazolide anion.

23. The method of claim 1 wherein E⁻ is a substituted or unsubstituted alkylsulfonate anion.

24. The method of claim 1 wherein E⁻ is methylsulfonate anion or trifluoromethylsulfonate anion.

25. The method of claim 1 wherein E⁻ is a substituted or unsubstituted arylsulfonate anion.

26. The method of claim 1 wherein E⁻ is a methylphenylsulfonate anion or a trihalomethylphenylsulfonate anion.

27. The method of claim 1 wherein E⁻ is trifluoromethylphenylsulfonate anion.

28. The method of claim 1 wherein E⁻ is tetrafluoroborate anion.

29. The method of claim 1 wherein E⁻ is hexafluorophosphate anion.

30. The method of claim 1 wherein E⁻ is a trihaloacetate anion.

31. The method of claim 1 wherein E⁻ is trifluoroacetate anion.

32. The method of claim 1 wherein D⁺ is aprotonated form of an alkyl amine.

33. The method of claim 1 wherein D⁺ is a protonated form of trimethyl amine, triethyl amine, triisopropyl amine, tributyl amine, triamyl amine, isopropyldimethyl amine, t-butyldimethyl amine, diisopropylethyl amine, or N,N,N′,N′-tetramethyl-1,2-diaminoethane.

34. The method of claim 1 wherein D⁺ is a protonated form of an aliphatic heterocyclic amine.

35. The method of claim 1 wherein D⁺ is a protonated form of any of DBU, N-methylmorpholine, N-methylpyrrolidine, N-methylpiperidine, N,N′-dimethylpiperazine, -ethylpyrrolidine, N-ethylpiperidine, N,N′-diethylpiperazine, 1,5-diazabicyclo[4.3.0]non-5-ene, 1,4-diazabicyclo[2.2.2]octane, or 1,5,7-triazabicyclo[4.4.0]dec-5ene.

36. The method of claim 1 wherein D⁺ is a protonated form of an aromatic heterocyclic amine.

37. The method of claim 1 wherein D⁺ is a protonated form of a mono-, di- or trialkyl pyridine that is optionally substituted with an amino group.

38. The method of claim 1 wherein D⁺ is a protonated form of any of 2,4,6-collidine, 2,6-lutidine, pyridine, 2-methylpyridine, 2,6-diethylpyridine, 2,6-di(t-butyl)pyridine, 4-methyl-2,6-di(t-butyl)pyridine, or 2,4,6-tri(t-butyl)pyridine.

39. The method of claim 1 wherein D⁺ is a protonated form of an alkylamino substituted pyridine.

40. The method of claim 1 wherein D⁺ is a protonated form of 4-dimethylaminopyridine.

41. The method of claim 1 wherein D⁺ is a protonated form of guanidine.

42. The method of claim 1 wherein D⁺ is a protonated form of N,N,N′N′-tetramethylguanidine.

43. The method of claim 1 wherein D⁺ is a quaternary tetraalkylammonium cation.

44. The method of claim 1 wherein D⁺ is a tetramethylammonium, tetraethylammonium, tetrapropylammonium, tetrabutylammonium, trimethyloctylammonium, or triethylbenzylammonium cation.

45. The method of claim 1 wherein E⁻ is a tetrazolide anion or substituted or unsubstituted alkylsulfonate anion, and D⁺ is a tetramethylammonium, tetraethylammonium, tetrapropylammonium, tetrabutylammonium, trimethyloctylammonium, or triethylbenzylammonium cation.

46. The method of claim 1 wherein E⁻ is trifluoromethanesulfonate anion and D⁺ is a protonated form of N-methylimidazole, N-ethylimidazole, or 1,2,4-triazole.

47. The method of claim 3 wherein D⁺ is a protonated form of trimethyl amine, triethyl amine, triisopropyl amine, tributyl amine, triamyl amine, isopropyldimethyl amine, t-butyldimethyl amine, diisopropylethyl amine, N,N,N′,N′-tetramethyl-1,2-diaminoethane, DBU, N-methylmorpholine, N-methylpyrrolidine, N-methylpiperidine, N,N′-dimethylpiperazine, N-ethylpyrrolidine, N-ethylpiperidine, N,N′-diethylpiperazine, 1,5-diazabicyclo[4.3.0]non-5-ene, 1,4-diazabicyclo[2.2.2]octane, or 1,5,7-triazabicyclo[4.4.0]dec-5ene, 2,4,6-collidine, 2,6-lutidine, pyridine, 2-methylpyridine, 2,6-diethylpyridine, 2,6-di(t-butyl)pyridine, 4-methyl-2,6-di(t-butyl)pyridine, or 2,4,6-tri(t-butyl)pyridine, 4-dimethylaminopyridine, or N,N,N′N′-tetramethylguanidine, or tetramethylammonium, tetraethylammonium, tetrapropylammonium, tetrabutylammonium, trimethyloctylammonium, or triethylbenzylammonium cation; and
E⁻ is 1H-tetrazolide anion, 4,5-dicyanoimidazolide anion, methylsulfonate anion, trifluoromethylsulfonate anion, methylphenylsulfonate anion, trifluoromethylphenylsulfonate anion, tetrafluoroborate anion, hexafluorophosphate anion, or trifluoroacetate anion.

48. A method of forming an internucleoside linkage comprising reacting a phosphoramidite of formula: 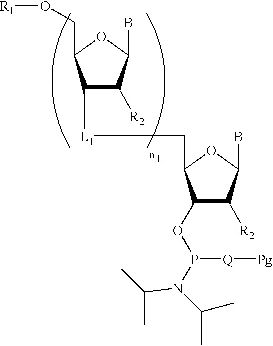
wherein:
L₁ is an internucleoside linkage;
n₁ is 0 to about 100;
R₁ is a hydroxyl protecting group;
R₂ is a 2′-substituent group;
R₄ and R₅ are each independently alkyl having from 1 to about 10 carbon atoms, or R₄ and R₅ taken together with the nitrogen atom to which they are attached form a heterocycle;
B is a nucleobase;
Q is O or S;
Pg is a phosphoryl protecting group;
with a compound of formula: 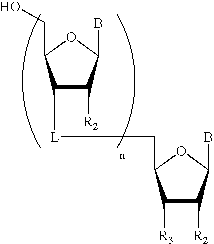
wherein
R₃ is a linker connected to a solid support;
n is from 1 to 100; and
L is an internucleoside linkage of formula: 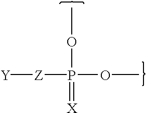
wherein:
Z is O or S;
X is O or S; and
Y is a phosphoryl protecting group or a negative charge;
provided that at least one Y is a negative charge;
wherein said reaction is performed in the presence of a neutralizing agent;
wherein said neutralizing agent is:
an aliphatic amine, an aliphatic heterocyclic amine, an aromatic amine, an aromatic heterocyclic amine, a guanidine, or a salt of formula D⁺E⁻ wherein:
D⁺ is a quaternary tetraalkylammonium cation, or a protonated form of an aliphatic amine, an aliphatic heterocyclic amine, an aromatic amine, an aromatic heterocyclic amine, or a guanidine; and
E⁻ is a tetrazolide anion, 4,5-dicyanoimidazolide anion, a substituted or unsubstituted alkylsulfonate anion, a substituted or unsubstituted arylsulfonate anion, tetrafluoroborate anion, hexafluorophosphate anion, or a trihaloacetate anion.

49. The method of claim 48 wherein said neutralizing agent is a salt of formula D⁺E⁻.

50. The method of claim 49 wherein E⁻ is a tetrazolide anion.

51. The method of claim 48 wherein E⁻ is 1 H-tetrazolide anion, 5-methylthio-1H-tetrazolide anion, 5-ethylthio-1H-tetrazolide anion or 1-phenyl-5-thiol-1H-tetrazolide anion.

52. The method of claim 48 wherein E⁻ is 1H-tetrazolide anion.

51. The method of claim 50 wherein D⁺ is a protonated form of any of an alkyl, alkenyl or alkynyl amine having from one to about 20 carbons, an aliphatic heterocyclic amine, an aromatic heterocyclic amine, or a guanidine.

52. The method of claim 48 wherein D⁺ is a protonated form of an alkyl amine.

53. The method of claim 50 wherein D⁺ is a protonated form of trimethyl amine, triethyl amine, triisopropyl amine, tributyl amine, triamyl amine, isopropyldimethyl amine, t-butyldimethyl amine, diisopropylethyl amine, or N,N,N′,N′-tetramethyl-1,2-diaminoethane.

54. The method of claim 50 wherein D⁺ is a protonated form of an aliphatic heterocyclic amine.

55. The method of claim 50 wherein D⁺ is aprotonated form of any of DBU, N-methylmorpholine, N-methylpyrrolidine, N-methylpiperidine, N,N′-dimethylpiperazine, -ethylpyrrolidine, N-ethylpiperidine, N,N′-diethylpiperazine, 1,5-diazabicyclo[4.3.0]non-5-ene, 1,4-diazabicyclo[2.2.2]octane, or 1,5,7-triazabicyclo[4.4.0]dec-5ene.

56. The method of claim 50 wherein D⁺ is a protonated form of an aromatic heterocyclic amine.

57. The method of claim 50 wherein D⁺ is a protonated form of a mono-, di- or trialkyl pyridine that is optionally substituted with an amino group.

58. The method of claim 50 wherein D⁺ is aprotonated form of any of 2,4,6-collidine, 2,6-lutidine, pyridine, 2-methylpyridine, 2,6-diethylpyridine, 2,6-di(t-butyl)pyridine, 4-methyl-2,6-di(t-butyl)pyridine, or 2,4,6-tri(t-butyl)pyridine.

59. The method of claim 50 wherein D⁺ is a protonated form of an alkylamino substituted pyridine.

60. The method of claim 50 wherein D⁺ is a protonated form of 4-dimethylaminopyridine.

61. The method of claim 50 wherein D⁺ is a protonated form of guanidine.

62. The method of claim 50 wherein D⁺ is a protonated form of a tetraalkyl guanidine.

63. The method of claim 50 wherein D⁺ is a protonated form of N,N,N′N′-tetramethylguanidine.

64. The method of claim 50 wherein D⁺ is a quaternary tetraalkylammonium cation.

65. The method of claim 50 wherein D⁺ is a tetramethylammonium, tetraethylammonium, tetrapropylammonium, tetrabutylammonium, trimethyloctylammonium, or triethylbenzylammonium cation.

66. The method of claim 50 wherein E⁻ is 1H-tetrazolide anion.

67. The method of claim 48 wherein E is 4,5-dicyanoimidazolide anion.

68. The method of claim 48 wherein E⁻ is a substituted or unsubstituted alkylsulfonate anion.

69. The method of claim 48 wherein E⁻ is methylsulfonate anion or trifluoromethylsulfonate anion.

70. The method of claim 48 wherein E⁻ is a substituted or unsubstituted arylsulfonate anion.

71. The method of claim 48 wherein E⁻ is a methylphenylsulfonate anion or a trihalomethylphenylsulfonate anion.

72. The method of claim 48 wherein E⁻ is trifluoromethylphenylsulfonate anion.

73. The method of claim 48 wherein E⁻ is tetrafluoroborate anion.

74. The method of claim 48 wherein E⁻ is hexafluorophosphate anion.

75. The method of claim 48 wherein E⁻ is a trihaloacetate anion.

76. The method of claim 48 wherein E⁻ is trifluoroacetate anion.

77. The method of claim 48 wherein D⁺ is a protonated form of an alkyl amine.

78. The method of claim 48 wherein D⁺ is a protonated form of trimethyl amine, triethyl amine, triisopropyl amine, tributyl amine, triamyl amine, isopropyldimethyl amine, t-butyldimethyl amine, diisopropylethyl amine, or N,N,N′,N′-tetramethyl-1,2-diaminoethane.

79. The method of claim 48 wherein D⁺ is a protonated form of an aliphatic heterocyclic amine.

80. The method of claim 48 wherein D⁺ is a protonated form of any of DBU, N-methylmorpholine, N-methylpyrrolidine, N-methylpiperidine, N,N′-dimethylpiperazine, -ethylpyrrolidine, N-ethylpiperidine, N,N′-diethylpiperazine, 1,5-diazabicyclo[4.3.0]non-5-ene, 1,4-diazabicyclo[2.2.2]octane, or 1,5,7-triazabicyclo[4.4.0]dec-5ene.

81. The method of claim 48 wherein D⁺ is a protonated form of an aromatic heterocyclic amine.

82. The method of claim 48 wherein D⁺ is a protonated form of a mono-, di- or trialkyl pyridine that is optionally substituted with an amino group.

83. The method of claim 48 wherein D⁺ is a protonated form of any of 2,4,6-collidine, 2,6-lutidine, pyridine, 2-methylpyridine, 2,6-diethylpyridine, 2,6-di(t-butyl)pyridine, 4-methyl-2,6-di(t-butyl)pyri dine, or 2,4,6-tri(t-butyl)pyridine.

84. The method of claim 48 whereinD⁺ is aprotonated form of an alkylamino substituted pyridine.

85. The method of claim 48 wherein D⁺ is a protonated form of 4-dimethylaminopyridine.

86. The method of claim 48 wherein D⁺ is a protonated form of guanidine.

87. The method of claim 48 wherein D⁺ is a protonated form of N,N,N′N′-tetramethylguanidine.

88. The method of claim 48 wherein D⁺ is a quaternary tetraalkylammonium cation.

89. The method of claim 48 wherein D⁺ is a tetramethylammonium, tetraethylammonium, tetrapropylammonium, tetrabutylammonium, trimethyloctylammonium, or triethylbenzylammonium cation.

90. The method of claim 48 wherein E⁻ is a tetrazolide anion or substituted or unsubstituted alkylsulfonate anion, and D⁺ is a tetramethylammonium, tetraethylammonium, tetrapropylammonium, tetrabutylammonium, trimethyloctylammonium, or triethylbenzylammonium cation.

91. The method of claim 48 wherein E⁻ is trifluoromethanesulfonate anion and D⁺ is a protonated form of N-methylimidazole, N-ethylimidazole, or 1,2,4-triazole.

92. The method of claim 50 wherein D⁺ is a protonated form of trimethyl amine, triethyl amine, triisopropyl amine, tributyl amine, triamyl amine, isopropyldimethyl amine, t-butyldimethyl amine, diisopropylethyl amine, N,N,N′,N′-tetramethyl-1,2-diaminoethane, DBU, N-methylmorpholine, N-methylpyrrolidine, N-methylpiperidine, N,N′-dimethylpiperazine, N-ethylpyrrolidine, N-ethylpiperidine, N,N′-diethylpiperazine, 1,5-diazabicyclo[4.3.0]non-5-ene, 1,4-diazabicyclo[2.2.2]octane, or 1,5,7-triazabicyclo[4.4.0]dec-5ene, 2,4,6-collidine, 2,6-lutidine, pyridine, 2-methylpyridine, 2,6-diethylpyridine, 2,6-di(t-butyl)pyridine, 4-methyl-2,6-di(t-butyl)pyridine, or 2,4,6-tri(t-butyl)pyridine, 4-dimethylaminopyridine, or N,N,N′N′-tetramethylguanidine, or tetramethylammonium, tetraethylammonium, tetrapropylammonium, tetrabutylammonium, trimethyloctylammonium, or triethylbenzylammonium cation; and
E⁻ is 1H-tetrazolide anion, 4,5-dicyanoimidazolide anion, methylsulfonate anion, trifluoromethylsulfonate anion, methylphenylsulfonate anion, trifluoromethylphenylsulfonate anion, tetrafluoroborate anion, hexafluorophosphate anion, or trifluoroacetate anion.

93. The method of claim 50 wherein Q is O; Z is O;
Pg is β-cyanoethyl, methyl, (N-methyl-N-benzoylamino)ethyl, (N-ethyl-N-benzoylamino)ethyl, 2-[-methyl-N-(4-methoxybenzoyl)amino]ethyl, 2-(N-isopropyl-N-benzoylamino)ethyl, 2-[N-ethyl-N-(4-methoxybenzoyl)amino]ethyl, 2-[N-isopropyl-N-(4 methoxybenzoyl)amino]ethyl, 2-[N-methyl-N-(4-dimethylaminobenzoyl)amino]ethyl, 2-[N-ethyl-N-(4-dimethylaminobenzoyl)amino]ethyl, 2-[N-isopropyl-N-(4-dimethylaminobenzoyl)amino]ethyl, 2-(thionobenzoylamino)ethyl, 3-(thionobenzoylamino)-propyl, 2-(N-phenylthiocarbamoylamino)ethyl, 2-[(1-naphthyl)carbamoyloxy]ethyl, diphenyl-silylethyl, δ-cyanobutenyl, cyanop-xylyl, methyl-N-trifluoroacetyl ethyl or acetoxy phenoxy ethyl; and
Y is β-cyanoethyl, allyl, methyl, (N-methyl-N-benzoylamino)ethyl, (N-ethyl-N-benzoylamino)ethyl, 2-[N-methyl-N-(4-methoxybenzoyl)amino]ethyl, 2-(N-isopropyl-N-benzoylamino)ethyl, 2-[N-ethyl-N-(4-methoxybenzoyl)amino]ethyl, 2-[N-isopropyl-N-(4-methoxybenzoyl)amino]ethyl, 2-[N-methyl-N-(4-dimethylaminobenzoyl)amino]ethyl, 2-[N-ethyl-N-(4-dimethylaminobenzoyl)amino]ethyl, 2-[N-isopropyl-N-(4-dimethylamino-benzoyl)amino]ethyl, 2-(thionobenzoylamino)ethyl, 3-(thionobenzoylamino)propyl, 2-(N-phenylthiocarbamoylamino)ethyl, 2-[(1-naphthyl)carbamoyloxy]ethyl, diphenylsilylethyl, 6-cyanobutenyl, cyano p-xylyl , methyl-N-trifluoroacetyl ethyl, acetoxy phenoxy ethyl, or a negative charge.

94. The method of claim 48 wherein:
said neutralizing agent is a salt of formula D⁺E⁻;
E⁻ is a tetrazolide anion;
D⁺ is a protonated form of a mono-, di- or trialkyl pyridine that is optionally substituted with an amino group;
Q is O;
Z is O;
R₄ and R₅ are each diisopropyl, or R₄ and R₅ together with the nitrogen atom to which they are attached form morpholine;
Pg is β-cyanoethyl, methyl, diphenylsilylethyl, δ-cyanobutenyl, cyano p-xylyl methyl-N-trifluoroacetyl ethyl or acetoxy phenoxy ethyl; and
Y is β-cyanoethyl, allyl, methyl, diphenylsilylethyl, δ-cyanobutenyl, cyano p-xylyl , methyl-N-trifluoroacetyl ethyl or acetoxy phenoxy ethyl or a negative charge.

95. The method of claim 94 wherein:
E⁻ is 1H-tetrazolide anion;
D⁺ is a protonated form of dimethylaminopyridine;
Pg is β-cyanoethyl, diphenylsilylethyl, δ-cyanobutenyl, cyanop-xylyl, methyl-N-trifluoroacetyl ethyl or acetoxy phenoxy ethyl; and
Y is β-cyanoethyl, allyl, diphenylsilylethyl, δ-cyanobutenyl, cyano p-xylyl, methyl-N-trifluoroacetyl ethyl, acetoxy phenoxy ethyl or a negative charge.

96. A method comprising the steps of:
(a) providing a solid support having a 5′-O-protected phosphorus-linked oligomer bound thereto, said phosphorus-linked oligomer having at least one phosphoryl internucleoside linkage that does not bear a phosphoryl protecting group;
(b) deprotecting the 5′-hydroxyl of the 5′-O-protected phosphorus-linked oligomer with a deprotecting reagent;
(c) washing the deprotected phosphorus-linked oligomer on the solid support with a solution containing a neutralizing agent;
(d) reacting the deprotected 5′-hydroxyl with an 5′-protected nucleoside phosphoramidite to produce a phosphite triester linkage therebetween; and
(e) oxidizing or sulfurizing the covalent linkage to form a phosphodiester, phosphorothioate, phosphorodithioate or H-phosphonate linkage; and
optionally repeating steps b through e at least once for subsequent couplings of additional nucleoside phosphoramidites;
wherein said neutralizing agent is:
an aliphatic amine, an aliphatic heterocyclic amine, an aromatic amine, an aromatic heterocyclic amine, a guanidine, or a salt of formula D⁺E⁻ wherein:
D⁺ is a quaternary tetraalkylammonium cation, or a protonated form of an aliphatic amine, an aliphatic heterocyclic amine, an aromatic amine, an aromatic heterocyclic amine, or a guanidine; and
E⁻ is a tetrazolide anion, 4,5-dicyanoimidazolide anion, a substituted or unsubstituted alkylsulfonate anion, a substituted or unsubstituted arylsulfonate anion, tetrafluoroborate anion, hexafluorophosphate anion, or a trihaloacetate anion.

97. A method comprising the steps of:
(a) providing a solid support having a 5′-O-protected phosphorus-linked oligomer bound thereto, said phosphorus-linked oligomer having at least one phosphoryl internucleoside linkage that does not bear a phosphoryl protecting group;
(b) deprotecting the 5′-hydroxyl of the 5′-O-protected phosphorus-linked oligomer with a deprotecting reagent to form a support bound 5′-deprotected phosphorus-linked oligomer;
(c) optionally washing the deprotected phosphorus-linked oligomer on the solid support;
(d) contacting the support bound 5′-deprotected phosphorus-linked oligomer with a solution comprising a 5′-protected nucleoside phosphoramidite to produce a phosphite triester linkage therebetween, wherein said solution further comprises a neutralizing agent; and
(e) oxidizing or sulfurizing the phosphite triester linkage to form a phosphodiester, phosphorothioate, phosphorodithioate or H-phosphonate linkage; and
optionally repeating steps b through e at least once for subsequent couplings of additional nucleoside phosphoramidites;
wherein said neutralizing agent is:
an aliphatic amine, an aliphatic heterocyclic amine, an aromatic amine, an aromatic heterocyclic amine, a guanidine, or a salt of formula D⁺E⁻ wherein:
D⁺ is a quaternary tetraalkylammonium cation, or a protonated form of an aliphatic amine, an aliphatic heterocyclic amine, an aromatic amine, an aromatic heterocyclic amine, or a guanidine; and
E⁻ is a tetrazolide anion, 4,5-dicyanoimidazolide anion, a substituted or unsubstituted alkylsulfonate anion, a substituted or unsubstituted arylsulfonate anion, tetrafluoroborate anion, hexafluorophosphate anion, or a trihaloacetate anion.

98. A composition comprising a 5′-protected nucleoside phosphoramidite and a salt of formula D⁺E⁻ wherein:
D⁺ is a quaternary tetraalkylammonium cation, or a protonated form of an aliphatic amine, an aliphatic heterocyclic amine, an aromatic amine, an aromatic heterocyclic amine, or a guanidine; and
E⁻ is a tetrazolide anion, 4,5-dicyanoimidazolide anion, a substituted or unsubstituted alkylsulfonate anion, a substituted or unsubstituted arylsulfonate anion, tetrafluoroborate anion, hexafluorophosphate anion, or a trihaloacetate anion.

99. The composition of claim 98 wherein:
E⁻ is a tetrazolide anion; and
D⁺ is a protonated form of a mono-, di- or trialkyl pyridine that is optionally substituted with an amino group.

100. The composition of claim 98 wherein:
E⁻ is 1H-tetrazolide anion; and
D⁺ is a protonated form of dimethylaminopyridine.

101. The composition of claim 98 further comprising a solid support having a 5′-O-protected phosphorus-linked oligomerbound thereto, said phosphorus-linked oligomer having at least one phosphoryl internucleoside linkage that does not bear a phosphoryl protecting group.

102. The composition of claim 99 further comprising a solid support having a 5′-O-protected phosphorus-linked oligomer bound thereto, said phosphorus-linked oligomer having at least one phosphoryl internucleoside linkage that does not bear a phosphoryl protecting group.

103. The composition of claim 100 further comprising a solid support having a 5′-O-protected phosphorus-linked oligomer bound thereto, said phosphorus-linked oligomer having at least one phosphoryl internucleoside linkage that does not bear a phosphoryl protecting group.
